(19) United States
(12) Patent Application Publication
(10) Pub. No.: US 2005/0192530 A1
(43) Pub. Date: Sep. 1, 2005

(54) Method and apparatus for needle-less injection with a degassed fluid

(75) Inventor: Thomas P. Castellano, Santa Monica, CA (US)

Correspondence Address: Pillsbury Winthrop LLP;Intellectual Property Group, Suite 2800, 725 South Figueroa Street, Los Angeles, CA, 90017-5406, US

(73) Assignee: PenJet Corporation, Santa Monica, CA (US)

(21) Appl. No.: 10/859,541

(22) Filed: Jun. 2, 2004

Related U.S. Application Data
(63) Continuation-in-part of application No. 10/227,885, filed on Aug. 26, 2002, now abandoned.
(63) Continuation-in-part of application No. 10/227,879, filed on Aug. 26, 2002, now abandoned.
Application No. 10/227,879 is a continuation of application No. 09/834,476, filed on Apr. 13, 2001, now Pat. No. 6,613,010.

(51) Int. Cl. A61M 5/30

(52) U.S. Cl. 604/70

(57) ABSTRACT

Apparatuses and methods are described for administering a needle-less injection of a degassed fluid. Prior to filling, or after filling but prior to administration of a needle-less injection, gas is removed from the fluid to create a degassed fluid. A needle-less injection may then be performed with a reduced risk of discomfort to the recipient of the injection and with lower potential for the creation of a subdermal hematoma as a result of the injection. A wide variety of needle-less injectors may be used in accordance with various embodiments of the present invention.

### RELATED APPLICATIONS

This application is a continuation-in-part of U.S. patent application Ser. No. 10/227,885, filed Aug. 26, 2002. This application is also a continuation-in-part of U.S. patent application Ser. No. 10/227,879, filed Aug. 26, 2002, which is a continuation of U.S. patent application Ser. No. 09/834,476, filed Apr. 13, 2001, now U.S. Pat. No. 6,613,010, issued Sep. 2, 2003.

This application is related to U.S. patent application Ser. No. 09/566,928, filed May 6, 2000, now U.S. Pat. No. 6,447,475, issued Sep. 10, 2002. Further, this application generally relates to U.S. patent application Ser. No. 09/215,769, filed Dec. 19, 1998, now U.S. Pat. No. 6,063,053, which is a continuation of U.S. patent application Ser. No. 08/727,911, filed Oct. 9, 1996, now U.S. Pat. No. 5,851,198, which is a continuation-in-part of U.S. patent application Ser. No. 08/719,459, filed Sep. 25, 1996, now U.S. Pat. No. 5,730,723, which is a continuation-in-part of U.S. patent application Ser. No. 08/541,470, filed Oct. 10, 1995, now abandoned. This application is also generally related to U.S. patent application Ser. No. 09/192,079, filed Nov. 14, 1998, now U.S. Pat. No. 6,080,130, and to U.S. patent application Ser. No. 09/808,511, filed Mar. 14, 2001, now U.S. Pat. No. 6,500,239, issued Dec. 31, 2002.

### FIELD OF THE INVENTION

This invention relates to needle-less injection apparatuses including a degassed fluid, and methods for performing a needle-less injection of a degassed fluid using the same.

### BACKGROUND OF THE INVENTION

Traditionally, fluids such as medications are injected into patients, either subdermally or intradermally, using hypodermic syringe needles. The body of the syringe is filled with the injectable fluid and, once the needle has pierced the patient's skin, the syringe plunger is depressed so as to expel the injectable fluid out of an opening in the needle. The person performing the injection is usually a trained medical services provider, who manually inserts the hypodermic needle between the layers of a patient's skin for an intradermal injection, or beneath the skin layers for a subcutaneous injection.

Intradermal or subdermal delivery of a medication through the use of a hypodermic needle requires some skill and training for proper and safe administration. In addition, the traditional method of intradermal injections requires actual physical contact and penetration of a needle through the skin surface of the patient, which can be painful for the patient. Traditional needle injectors, such as hypodermic syringes, are also expensive to produce and difficult to use with prepackaged medication doses. Needle injectors also suffer from increased danger of contamination exposure to health care workers administering the injections, and to the general public when such injectors are not properly disposed of.

Jet injectors are generally designed to avoid some or all of these problems. However, not only are conventional jet injectors cumbersome and awkward, but, existing conventional jet injectors are only capable of subcutaneous delivery of a medication beneath the skin layers of a patient. Conventional jet injectors are also somewhat dangerous to use, since they can be discharged without being placed against the skin surface. With a fluid delivery speed of about 800 feet per second (fps) and higher, a conventional jet injector could injure a person's eye at a distance of up to 15 feet. In addition, jet injectors that have not been properly sterilized are notorious for creating infections at the injection site. Moreover, if a jet injector is not positioned properly against the injection site, the injection can result in wetting on the skin surface. Problems associated with improper dosage amounts may arise as well, if some portion of the fluid intended for injection remains on the skin surface following an injection, having not been properly injected into and/or through the skin surface.

Subdermal hematomas, tissue damage, and scarring from mechanical force injury may result from the use of needle-less injectors when pockets of gas are present in the injector ampoule prior to dispensing the medication contained therein. Within the 800 to 1200 foot per second range, optimal for acceleration of liquid medication through the skin via a needle-less injector, liquid readily penetrates the skin while air does not. Thus, gas pockets accelerated against the skin lead to the formation of a bruise and can be quite painful for the recipient, whereas liquid medication passes into and/or through the skin without discomfort.

In general, the gas pocket is found at the dispensing terminus of the ampoule, which is proximate to the skin, though this can change depending on the orientation of the ampoule during storage. Further, when a cap is removed from the end of a needle-less injector, exposing the dispensing area for application to the skin surface, any gas pocket not already situated at the dispensing end may tend to migrate toward that end, due to the pressure change caused by cap removal. This motion of the gas pocket often forces some liquid from the ampoule, thereby diminishing the volume of liquid that will be injected into the recipient. This renders the dosage level inaccurate, as a nontrivial volume of medication is lost from the injector prior to use.

Gas pockets may be present from the outset, resulting from improper filling of an ampoule. Filling the ampoule with an insufficient amount of liquid clearly leaves such a pocket. However, overfilling the ampoule and removing any excess to arrive at the desired volume is generally not a practical alternative, since it is likely that a small amount of liquid will remain on the outer surface of the ampoule. In the medical context, any such liquid is likely to foster the growth of bacteria, which is unacceptable in a scenario where sterile conditions are imperative. Any ampoule with such bacterial growth must be disposed of, and is therefore wasteful.

Even in a perfectly filled ampoule, where no cognizable gas pockets are present immediately following loading, pockets may still develop over time as the dissolved gases present in the liquid separate out from solution. Dissolved gases are present in the liquids filled into ampoules under normal conditions (i.e., wherein filling is not performed in a vacuum, or the like) in concentrations proportional to their partial pressure in air. These dissolved gases consist mostly of nitrogen and oxygen, along with several trace gases, and are found latent in the solution in amounts related to their partial pressures in the local atmosphere.

The size of gas pockets varies according to the pharmaceutical active in solution, as some actives allow liquid to retain greater amounts of gas than others, but in some instances a pocket may be as large as 20% of the total ampoule volume. This naturally occurring formation of gas pockets is exacerbated when pre-filled ampoules remain unused for substantial periods of time. Again, varying with the type of active in solution, some actives will form substantial gas pockets after only a few days, while others may not form a pocket for a year or more. For certain medicaments, an ampoule may be stored as long as three to five years, and nearly every active will generate a gas pocket in that amount of time.

Increased temperature also effects the separation of gas from solution, prompting gas pockets to form faster and larger. However, pharmaceutical actives generally require storage within a certain optimal temperature range in order to prevent the active from breaking down and thus losing efficacy; this temperature range being determined independently of the potential for separation of gas from solution. For example, many proteins suitable for injection will denature at high temperatures or will lose potency when excessively chilled. Since optimal temperature ranges for efficacy may not have any correlation with a temperature that would avoid a gas pocket from forming in storage, one may be forced to choose between either preserving drug efficacy or minimizing gas pocket formation.

In the context of injection by more traditional means such as with a preloaded syringe, it is well established that any significant amount of air in such a device will cause pain for the recipient and potentially far more dire consequences if the amount of air is substantial. Gas pockets may develop in these syringes much in the way described above with regard to ampoules of needle-less injectors, as these devices are frequently subject to similar storage conditions and requirements. Those administering such injections can more readily obviate these limitations, however, as air may be evacuated from the liquid-containing chamber of a syringe by partially depressing the plunger while the syringe is inverted immediately prior to administration of an injection. This is generally not possible with a needle-less injector, as the entire volume of a needle-less injector ampoule is evacuated in one step during normal operation. Moreover, liquid that is inadvertently evacuated from the chamber of a syringe along with the undesirable air does not present a sterility concern, since bacteria will not grow in a pharmacologically hazardous amount in the few moments between evacuating such air and administering an injection.

Examples of needle-less injectors may include, but are in no way limited to, those described in the following:
U.S. Pat. No. 6,673,034, issued Jan. 6, 2004, U.S. Pat. No. 6,447,475, issued Sep. 10, 2002, U.S. Pat. No. 6,063,053, issued May 16, 2000, U.S. Pat. No. 5,851,198, issued Dec. 22, 1998, U.S. Pat. No. 5,730,723, issued Mar. 24, 1998, and U.S. Pat. No. 6,080,130, issued Jun. 27, 2000, each to PenJet Corporation;
U.S. patent application publication No. 2001/0039394 A1, filed Dec. 24, 1998, U.S. Pat. No. 6,135,979, issued Oct. 24, 2000, U.S. Pat. No. 5,957,886, issued Sep. 28, 1999, U.S. Pat. No. 5,891,086, issued Apr. 6, 1999 and U.S. Pat. No. 5,480,381, issued Jan. 2, 1996, each to Weston Medical Limited;
U.S. Pat. No. 6,383,168 B1, issued May 7, 2002, U.S. Pat. No. 6,319,224 B1, issued Nov. 20, 2001, U.S. Pat. No. 6,264,629 B1, issued Jul. 24, 2001, U.S. Pat. No. 6,132,395, issued Oct. 17, 2000, U.S. Pat. No. 6,096,002, issued Aug. 1, 2000, U.S. Pat. No. 5,993,412, issued Nov. 30, 1999, U.S. Pat. No. 5,520,639, issued May 28, 1996, U.S. Pat. No. 5,064,413, issued Nov. 12, 1991, U.S. Pat. No. 4,941,880, issued Jul. 17, 1990, U.S. Pat. No. 4,790,824, issued Dec. 13, 1988 and U.S. Pat. No. 4,596,556, issued Jun. 24, 1986, each to Bioject, Inc.;
U.S. Pat. No. 6,168,587 B1, issued Jan. 2, 2001, and U.S. Pat. No. 5,899,880, issued May 4, 1999, each to Powderject Research Limited;
U.S. Pat. No. 5,704,911, issued Jan. 6, 1998, and U.S. Pat. No. 5,569,189, issued Oct. 29, 1996, each to Equidyne Systems, Inc.;
U.S. Pat. No. 5,024,656, issued Jun. 18, 1991, and U.S. Pat. No. 4,680,027, issued Jul. 14, 1987, each to Injet Medical Products, Inc.;
U.S. Pat. No. 6,210,359 B1, issued Apr. 3, 2001, to Jet Medica, L.L.C.;
U.S. Pat. No. 6,406,455 B1, issued Jun. 18, 2002, to BioValve Technologies, Inc.; and
U.S. Pat. No. 5,891,085, issued Apr. 6, 1999, and U.S. Pat. No. 5,599,302, issued Feb. 4, 1997, each to Medi-Ject Corporation.

### SUMMARY OF THE DISCLOSURE

It is therefore an object of an embodiment of the instant invention to provide gas-pressured needle-less injectors that obviate, for practical purposes, the above-mentioned limitations.

The present invention relates to apparatuses and methods for administering a needle-less injection of a degassed fluid. The fluid may be degassed by any number of methods, such as any of those described in U.S. patent application Ser. No. 09/808,511, filed Mar. 14, 2001, now U.S. Pat. No. 6,500,239, issued Dec. 31, 2002, the disclosure of which is incorporated by reference herein. Other methods of degassing the fluid of the present invention may be apparent to one of skill in the art, and are contemplated as being within the scope of the present invention. The degassed fluid may be administered to a recipient with a needle-less injector that contains the degassed fluid prior to administration of an injection.

### BRIEF DESCRIPTION OF THE DRAWINGS

FIGS. 1a-1e illustrate a needle-less injector in accordance with an embodiment of the instant invention. FIG. 1a is a side perspective view prior to administration of an injection, shown at 0° rotation about the central axis of the injector, FIG. 1b is a side cross-sectional view, the injector having been rotated 90° about the central axis, FIG. 1c is a side perspective view at 0° rotation about the central axis, FIG. 1d is a side perspective view after administration of an injection, shown at 180° rotation about the central axis of the injector and FIG. 1e is a side partial cross-sectional view after administration of an injection, the injector having been rotated 90° about the central axis.

FIGS. 2a-2c illustrate the housing of a needle-less injector in accordance with an embodiment of the instant invention. FIG. 2a is a side perspective view at 180o rotation about the central axis of the injector, FIG. 2b is a proximate end perspective view and FIG. 2c is a distal end perspective view.

FIGS. 3a-c illustrate the ampoule cap of a needle-less injector in accordance with an embodiment of the instant invention. FIG. 3a is a side perspective view, FIG. 3b is a side cross-sectional view and FIG. 3c is a proximate end perspective view.

FIGS. 4a-c illustrate the plunger of a needle-less injector in accordance with an embodiment of the instant invention. FIG. 4a is a side perspective view, FIG. 4b is a side cross-sectional view and FIG. 4c is a proximate end perspective view.

FIGS. 5a-d illustrate the piston of a needle-less injector in accordance with an embodiment of the instant invention. FIG. 5a is a side perspective view, FIG. 5b is a side cross-sectional view, FIG. 5c is a proximate end perspective view and FIG. 5d is a distal end perspective view.

FIGS. 6a-d illustrate the diffuser of a needle-less injector in accordance with an embodiment of the instant invention. FIG. 6a is a side perspective view, FIG. 6b is a side cross-sectional view, FIG. 6c is a proximate end perspective view and FIG. 6d is a distal end perspective view.

FIGS. 7a-i illustrate various configurations of channels in the diffuser of a needle-less injector in accordance with embodiments of the instant invention.

FIGS. 8a-d illustrate the trigger of a needle-less injector in accordance with an embodiment of the instant invention. FIG. 8a is a side perspective view at 0o rotation about the central axis of the trigger, FIG. 8b is a side cross-sectional view at 90o rotation, FIG. 8c is a proximate end perspective view and FIG. 8d is a distal end perspective view.

FIGS. 9a-b illustrate the safety clamp of a needle-less injector in accordance with an embodiment of the instant invention. FIG. 9a is a proximate end perspective view and FIG. 9b is a side perspective view.

FIGS. 10a-d illustrate the engine housing of a needle-less injector in accordance with an embodiment of the instant invention. FIG. 10a is a side perspective view, FIG. 10b is a side cross-sectional view, FIG. 10c is a proximate end perspective view and FIG. 10d is a distal end perspective view.

FIGS. 11a-d illustrate the valve body of a needle-less injector in accordance with an embodiment of the instant invention. FIG. 11a is a side perspective view, FIG. 11b is a side cross-sectional view and FIG. 11c is a proximate end perspective view.

FIGS. 12a-c illustrate the closing ferrule of a needle-less injector in accordance with an embodiment of the instant invention, prior to the closing ferrule being mechanically fitted around a valve body and an engine housing. FIG. 12a is a side perspective view, FIG. 12b is a side cross-sectional view and FIG. 12c is a proximate end perspective view.

FIGS. 13a-d illustrate the threaded valve stem guide of a needle-less injector in accordance with an embodiment of the instant invention. FIG. 13a is a side perspective view in partial cross-section, FIG. 13b is a side cross-sectional view, FIG. 13c is a proximate end perspective view and FIG. 13d is a distal end perspective view.

FIGS. 14a-c illustrate the valve stem of a needle-less injector in accordance with an embodiment of the instant invention. FIG. 14a is a side perspective view, FIG. 14b is a side cross-sectional view prior to the distal end being shaped and FIG. 14c is a proximate end perspective view.

FIGS. 15a-b illustrate the valve spring of a needle-less injector in accordance with an embodiment of the instant invention. FIG. 15a is a side perspective view in the relaxed state, FIG. 15b is a side perspective view in the compressed state.

FIG. 16 is a graph depicting the velocity of the driver of an embodiment of the instant invention during administration of an injection.

FIGS. 17-25 depict aspects of a needle-less injector in accordance with an embodiment of the present invention. The needle-less injector depicted therein includes a cannula that pierces a membrane of a gas chamber.

FIGS. 26-36 depict aspects of a needle-less injector in accordance with an embodiment of the present invention. The needle-less injector depicted therein includes a latch to initiate an injection.

FIGS. 37-42 depict aspects of a needle-less injector in accordance with an embodiment of the present invention. The needle-less injector depicted therein is battery powered.

FIGS. 43-56 depict aspects of a needle-less injector in accordance with an embodiment of the present invention. The needle-less injector depicted therein includes a drive control mechanism.

FIGS. 57-66 depict aspects of a needle-less injector in accordance with an embodiment of the present invention. The needle-less injector depicted therein includes a lyophilized product.

### DETAILED DESCRIPTION OF THE PREFERRED EMBODIMENTS

As shown in the drawings for purposes of illustration, the invention is embodied in apparatuses and methods for administering a needle-less injection of a degassed fluid. In preferred embodiments of the present invention, use of the system and method avoid or minimize the formation of subdermal hematomas (bruising) from a needle-less injection, and further avoid the formation of a gas pocket in an ampoule of a needle-less injector or other suitable container filled with fluid.

The apparatuses and methods of the present invention may be used in conjunction with any needle-less injector. Needle-less injectors may include, but are in no way limited to, single use needle-less injectors that are either pre-filled with a fluid and stored for any period of time or filled with a fluid immediately prior to administration of a needle-less injection; reusable needle-less injectors that include a sufficient quantity of a fluid to administer multiple injections in series, to multiple recipients without need for refilling and those that must be refilled for each administration of an injection therewith; needle-less injectors that have a separate ampoule component that may be filled and stored separate from the remainder of the injector and those which are unitary needle-less injectors (e.g., those which include a housing that acts as an ampoule); and needle-less injectors that are powered by a spring, by gas pressure or, at least in part, by electricity. Needle-less injectors may be configured in a variety of ways; several examples are described in the U.S. patents and patent applications enumerated above, the disclosures of which are incorporated herein by reference, and in the ensuing Examples.

The degassed fluid appropriate for use in accordance with the apparatuses and methods of the instant invention may include any liquids, solutions, suspensions, mixtures, diluents, reagents, solvents (e.g., for mixing with a lyophilized product to create an injectable solution), emulsions, pharmaceutical vehicles or excipients, or other fluids that contain a gas, such as a dissolved gas, prior to a degassing operation. In preferred embodiments, the degassed fluid is selected from those appropriate for injection with any needle-less injector. Such fluids may include, but are not limited to, vaccines, injectable medications, drugs, pharmaceutical agents, nucleotide based (e.g., DNA, RNA) medications, saline solution, non-medicinal fluids administered as a placebo in a clinical study and the like. Preferably, in those embodiments of the present invention wherein a solute is dissolved in the fluid, the molecular weight of the solute is preferably in the range of from about 1 to about 500,000 Daltons. Accordingly, in these embodiments, the viscosity of the fluid may generally be in the range of from about 0.2 to about 10 Centipoise. Preferably, the viscosity of the fluid is in the range of from about 0.4 to about 2.0 Centipoise.

A degassing operation may include any operation performed to remove at least a portion of the dissolved gas from a fluid. Preferably, a substantial portion of the dissolved gas may be removed from the fluid by the degassing operation, although in some circumstances the complete or near complete removal of dissolved gas may not be readily achieved. In a most preferred embodiment, the amount of dissolved gas removed from a fluid is an amount which reduces the potential for the formation of an air or gas bubble in a pre-filled needle-less injector during storage. Any fluid which has been at least partially degassed is contemplated as being within the scope of “degassed fluids” as used herein, even if a less than optimum amount of gas has been removed therefrom, and even if the degassing operation is determined to be only partially successful.

### EXAMPLES

The following examples describe various needle-less injectors that may be suitable for use in accordance with the apparatuses and methods of the present invention. A wide variety of needle-less injectors may be used in the present invention, and the following needle-less injectors are intended only as examples of such injectors, and not as a complete listing of those which may be suitable.

### Example 1

### Modular Gas-Pressured Needle-Less Injector

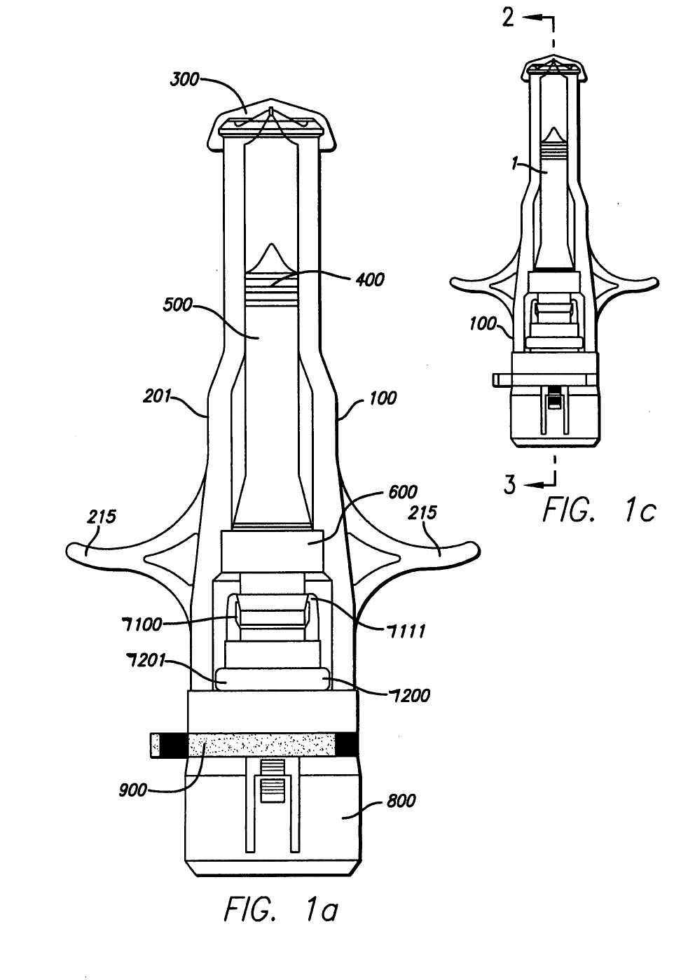

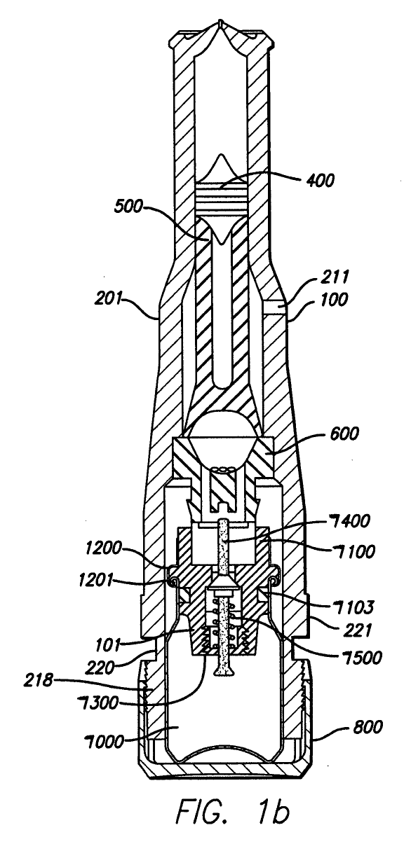

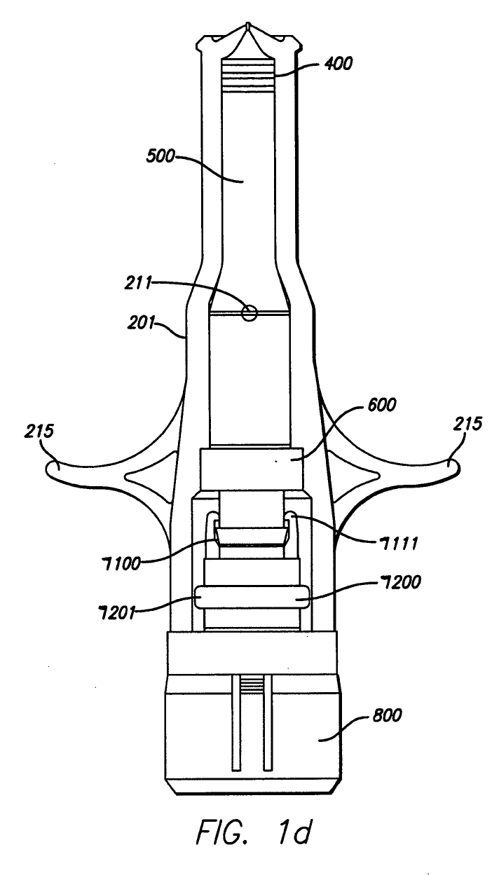

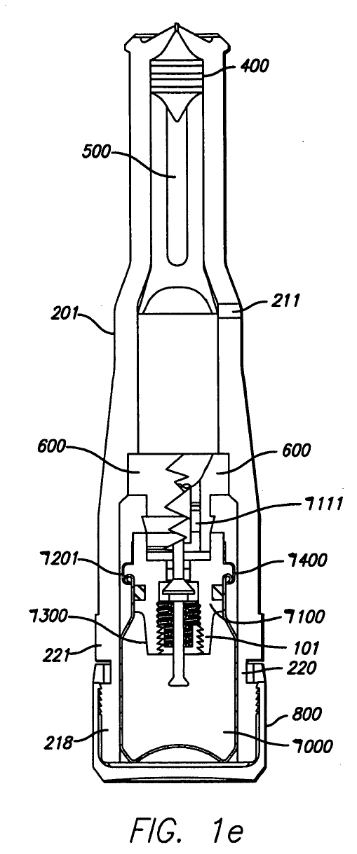

For ease in describing the various elements of the modular gas-pressured needle-less injector, the following spatial coordinate system will apply thereto. As depicted in FIG. 1c, a central axis is defined through the length of a gas-pressured needle-less injector 100. This central axis 1 has one terminus at the proximate end 2 of the needle-less injector 100, defined as that end of the device in contact with an injection surface during normal operation of the injector. The other terminus of the central axis is at the distal end 3 of the injector 100, defined as that end of the device furthest from the injection surface when the injector is positioned perpendicular to the injection surface. Thus, various elements of the device of the instant invention may be described with reference to their respective proximate and distal portions, as well as their central axes.

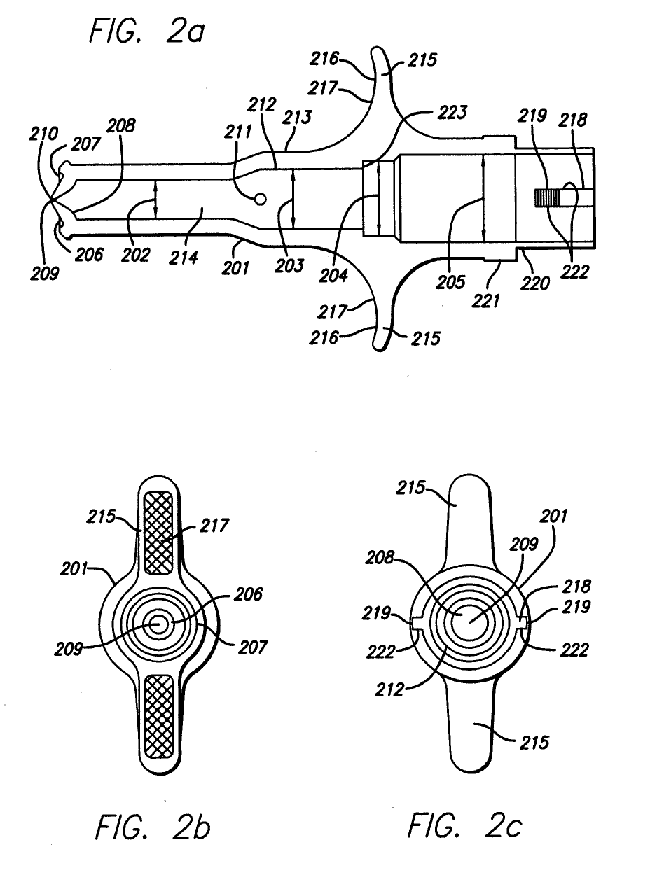

As depicted in FIG. 1, a gas-pressured needle-less injector 100 includes a housing 201. The housing 201 may be of any suitable shape, though in preferred embodiments it is roughly cylindrical about the central axis. The housing 201 preferably has a varying interior diameter along its length to accommodate the elements that reside and operate therein when the injector 100 is fully assembled. The housing 201 depicted in FIG. 2a has four such interior diameters: an ampoule diameter 202, a piston diameter 203, a diffuser diameter 204 and an engine diameter 205, respectively. Embodiments of the instant invention preferably do not have an ampoule that is a mechanical element separate and distinct from the housing 201, yet the housing 201 may act as an ampoule for various purposes such as filling with degassed fluid.

The exterior portion 206 of the proximate end surface of the housing 201 may be flat, though in preferred embodiments it is of a shape that maximizes injector efficacy. Efficacy is optimal when substantially all degassed fluid contained in the injector 100 is delivered through the injection surface, leaving substantially no degassed fluid on either the injection surface or the exterior portion 206 of the proximate end surface of the housing 201 after an injection is complete (see FIGS. 1d and 1e). To that end, in the embodiment depicted in FIG. 2a, the exterior portion 206 of the proximate end of the housing 201 is adapted to pinch and stretch the surface through which an injection is to be administered, as the exterior portion 206 of the proximate end surface of the housing 201 is brought into contact with an injection surface. Thus, the exterior portion 206 of the proximate end of the housing 201 preferably has a conical shape about the central axis, and further possesses an elevated rim 207 around its circumference.

The interior portion 208 of the proximate end of the housing 201 may be of any appropriate shape. It may conform roughly to the shape of the exterior portion 207, or have a design independent thereof. In one embodiment, the interior portion 208 is flat, though preferably, as depicted in FIG. 2a the interior portion 208 is roughly conical, with at least one orifice 209 at or near the vertex 210. The needle-less injector 100 depicted in FIG. 1 is shown with only one orifice.

The at least one orifice 209 provides fluid communication between the interior 214 of the housing 201 and the surface through which an injection is administered. The number of orifices 209 may be varied depending on the delivery parameters of the degassed fluid to be injected. One such parameter is the depth to which a degassed fluid must penetrate a recipient's tissue, when the device is used for the injection of a medicament into a human being. For example, in one embodiment it may be desirable to inject a degassed fluid just beneath the outermost skin layers of a recipient, and multiple orifices may best suit that end. Alternatively, a single orifice may be most desirable for an injection that requires deeper penetration for maximum drug efficacy.

An exhaust passage 211 may be created through the housing 201, from the interior wall 212 to the exterior wall 213, preferably within the section of the housing 201 of ampoule diameter 202. The exhaust passage 211 allows gas to vent from the interior 214 of the housing 201 preferably only after an injection has been administered. Thus, most preferably, the exhaust passage 211 is located at a point in the housing 201 at or immediately distal to the location of the piston 500 (see FIGS. 1d and 1e) after administration of an injection. In these most preferred embodiments, gas may not vent from the interior 214 of the housing 201 through the exhaust passage 211 until after substantially all degassed fluid contained in the housing 201 has been discharged from the needle-less injector 100, with the piston 500 at rest in its final position.

Degassed fluid stored in the needle-less injector 100, prior to administration of an injection, is preferably contained in the interior 214 of the housing 201 in the region bounded by the interior portion 208 of the proximate end of the housing 201, the interior wall 212 of the housing 201 and the proximate end 403 of the plunger 400 (see FIGS. 1a and 2a).

As depicted in FIG. 2a, the housing 201 may further include finger rests 215. In preferred embodiments, two such finger rests 215 are formed on the exterior wall 213 of the housing 201 at opposing locations. Most preferably, the finger rests 215 are located directly opposite one another. In preferred embodiments, each finger rest 215 has an arc 216 on the proximate side thereof to accommodate proper finger placement for either self-administration of an injection or assisted administration by a health care professional or the like. In the most preferred embodiments, the arcs 216 of the finger rests 215 further contain a non-slip, textured surface 217.

When the needle-less injector 100 is used by an individual performing self-administration of an injection, the individual's thumb and middle finger may be placed in the arcs 216 of the finger rests 215 on opposing sides of the housing 201 for stabilization of the device, with the index finger operably placed against the trigger 800 at the distal end of the injector 100. Another manner in which a user may perform self-administration of an injection, which is also the manner preferred when the needle-less injector 100 is operated by an individual other than the recipient of an injection, involves the index and middle fingers being placed in the arcs 216 of the finger rests 215 on opposing sides of the housing 201 for stabilization of the device, with the thumb operably placed against the trigger 800 at the distal end of the injector 100.

The housing 201 may further contain at least one latch retainer mechanism 218 near the distal end. The at least one latch retainer mechanism 218 may be comprised of a single set of saw tooth ridges that encircle the exterior wall 213 of the housing 201 around its central axis. More preferably, there are two latch retainer mechanisms 218 comprising two sets of saw tooth ridges 219, disposed opposite one another on the exterior wall 213 of the housing 201, though any appropriate number of latch retainer mechanisms 218 may be utilized. Preferably, as shown in FIG. 1b, the housing 201 further contains a clamp indentation 220 that is defined on its proximate end by a ridge 221 and on its distal end by the at least one latch retainer mechanism 218 and the proximate end of the trigger 800.

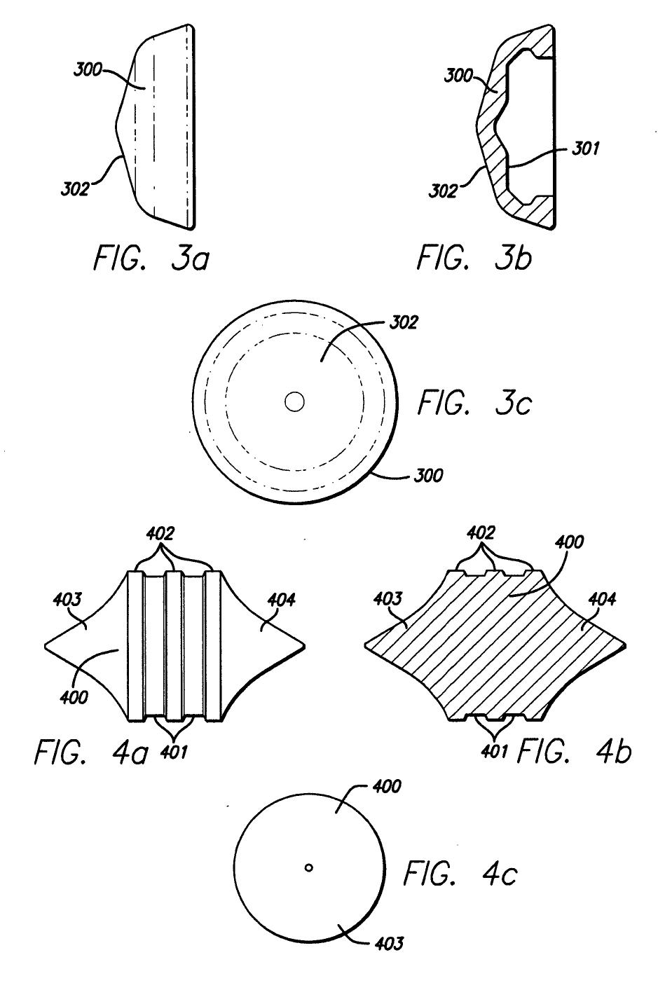

The proximate end of the housing 201 may further be fit with an ampoule cap 300, as depicted in FIG. 3, which serves to maintain sterility of the exterior portion 206 of the proximate end surface of the housing 201 while the needle-less injector 100 is stored. Further, when degassed liquids are used in accordance with the present invention, the ampoule cap 300 provides the requisite airtight seal between the at least one orifice 209 in the proximate end of the housing 201 and the local atmosphere, such that the degassed liquids may remain gas-free during storage. Referring again to FIG. 3, the interior 301 of the ampoule cap 300 is preferably designed to conform substantially to the exterior surface 206 of the proximate end of the housing 201, while the exterior 302 of the ampoule cap 300 may be of any convenient configuration.

As depicted in FIG. 4, the housing 201 may be fit with a plunger 400. Preferably, the plunger 400 is pressure-fit within the housing 201, as its diameter is equivalent to or slightly greater than the ampoule diameter 202 of the housing 201. The plunger 400 is preferably constructed of a sufficiently elastic material such that the pressure-fit creates an air and liquid-tight seal with the interior wall 212 of the housing 201. The plunger 400 is preferably cylindrical to mirror the shape of the interior wall 212 of the housing 201, though other shapes may be suitable especially where the interior wall 212 of the housing 201 is not cylindrical. Moreover, the wall 401 of the plunger 400 may have multiple ridges 402 disposed thereupon. Preferably, there are at least two such ridges 402, and most preferably there are three ridges 402. These ridges 402 provide stability to the plunger 400 such that its direction of travel during administration of an injection remains substantially linear along the central axis, without rotational motion around any axis other than the central axis.

The proximate end 403 of the plunger 400 may be of any suitable shape, including a flat surface, though in preferred embodiments it roughly mirrors the shape of the interior wall 208 of the proximate end of the housing 201. However, the elastic properties of the plunger material may allow the proximate end 403 of the plunger 400 to conform to the shape of a surface different than its own when mechanically forced against such a surface. Thus, the shape of the proximate end 403 of the plunger 400 need not mirror the shape of the interior wall 208 of the proximate end of the housing 201, yet the plunger proximate end 403 may conform to the shape of the interior wall 208 when forced against it during or after an injection is administered. In most preferred embodiments, however, the proximate end 403 of the plunger 400 is roughly conical in shape.

The distal end 404 of the plunger 400 may similarly be of any suitable shape, and is received by the proximate end of the piston 500. In preferred embodiments, the plunger 400 is symmetrical in shape along a plane perpendicular to the central axis. Thus, in preferred embodiments, the distal end 404 of the plunger 400 is roughly conical in shape.

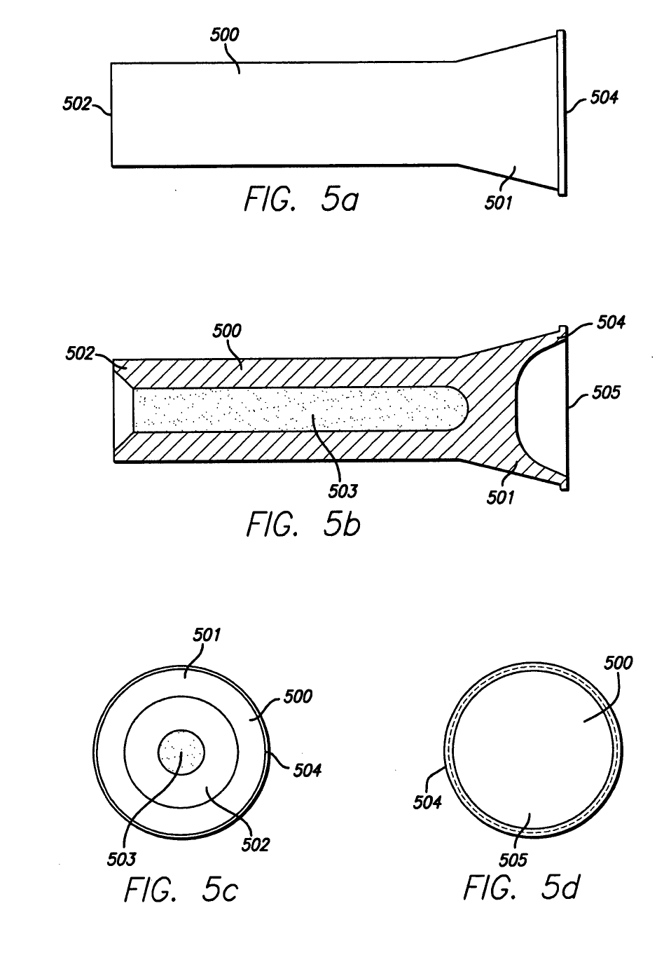

The housing 201 may be fit with a piston 500, as depicted in FIG. 5. The piston 500 preferably is of roughly cylindrical shape along the length of its central axis with a flared portion 501 toward its distal end, though other shapes may be appropriate especially in those embodiments where the interior wall 212 of the housing 201 is non-cylindrical. Preferably, the proximate end 502 of the piston 500 is shaped such that it mechanically receives the distal end 404 of the plunger 400. Thus, in most preferred embodiments, the proximate end 502 of the piston 500 is a roughly conical indentation. In preferred embodiments, the piston 500 further includes a chamber 503 that extends from the vertex of the conical indentation 502 along the central axis of the piston 500.

The exterior of the distal section of the piston is preferably a flared portion 501, terminating in an expansion cup rim 504. In most preferred embodiments, the distal section of the piston further has a hollow expansion cup 505. This expansion cup 505 is not in gaseous communication with the chamber 503 that extends from the proximate end 502 of the piston 500 along the piston central axis, as the chamber 503 does not extend entirely through the piston 500 to the expansion cup 505.

Referring to FIGS. 2a and 5, the distal section of the piston 500 may be pressure-fit within the portion of the housing 201 of piston diameter 203, such that the diameter of the expansion cup rim 504 of the piston 500 is substantially equivalent to the piston diameter 203 of the housing 201. Alternatively, the diameter of the expansion cup rim 504 may be slightly less than the piston diameter 203 of the housing 201. During use of the needle-less injector 100, the expansion cup 505 may expand radially due to the force of compressed gas pushing upon it. This serves to optimize the performance of the piston 500, as a substantially airtight seal is thus formed between the expansion cup rim 504 and the interior wall 212 of the housing 201.

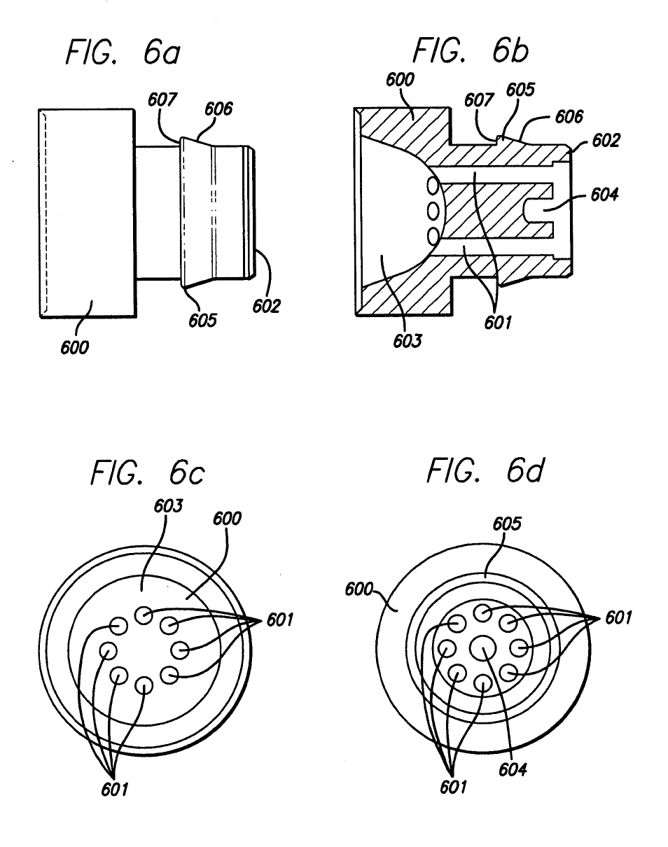

The housing 201 may be fit with a diffuser 600, as depicted in FIG. 6. The diffuser 600 is preferably affixed to the housing 201 along the interior wall 212 thereof at the portion of diffuser diameter 204. Affixing may be performed by high frequency welding or other suitable means. Most preferably, the diffuser 600 is affixed to the housing 201 only after the plunger 400 and piston 500 have been fit within the housing 201.

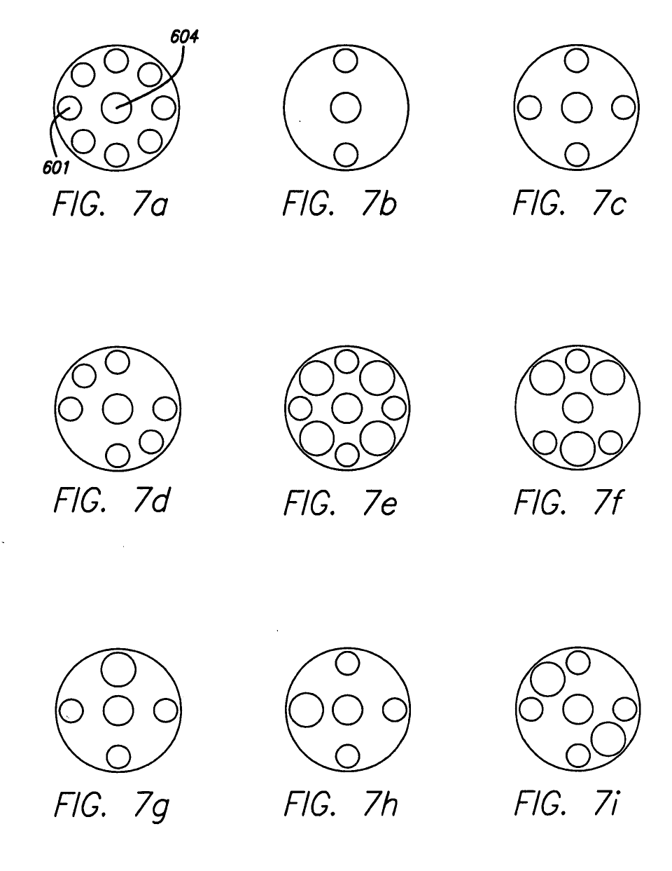

The diffuser 600 preferably further contains at least one channel 601 that provides gaseous communication between the distal end 602 of the diffuser 600 and the base of the diffuser cup 603. The at least one channel 601 is sized and positioned to optimize the injection delivery parameters of a particular degassed fluid. In preferred embodiments, as illustratively depicted in FIG. 7, the diffuser 600 may contain between two and eight channels 601, which may be of the same or different diameter, and may be symmetrically or non-symmetrically oriented about the central axis of the diffuser 600. Selection of various combinations of channels 601 in the diffuser 600 will affect the delivery performance of the needle-less injector 100, altering, for example, the initial acceleration of the driver of the needle-less injector 100. The velocity of the driver of the preferred embodiment of the instant invention is depicted in FIG. 16. Notably, the compressed gas engine of the instant invention allows for a substantially constant delivery velocity during the bulk of the injection.

Referring to FIG. 6b, a valve stem support depression 604 may further be included on the distal end 602 of the diffuser 600, located at the diffuser central axis. The diffuser 600 may further contain a locking ring 605 around its outer circumference. Preferably the locking ring 605 is angled on its distal surface 606, but is flat on its proximate surface 607.

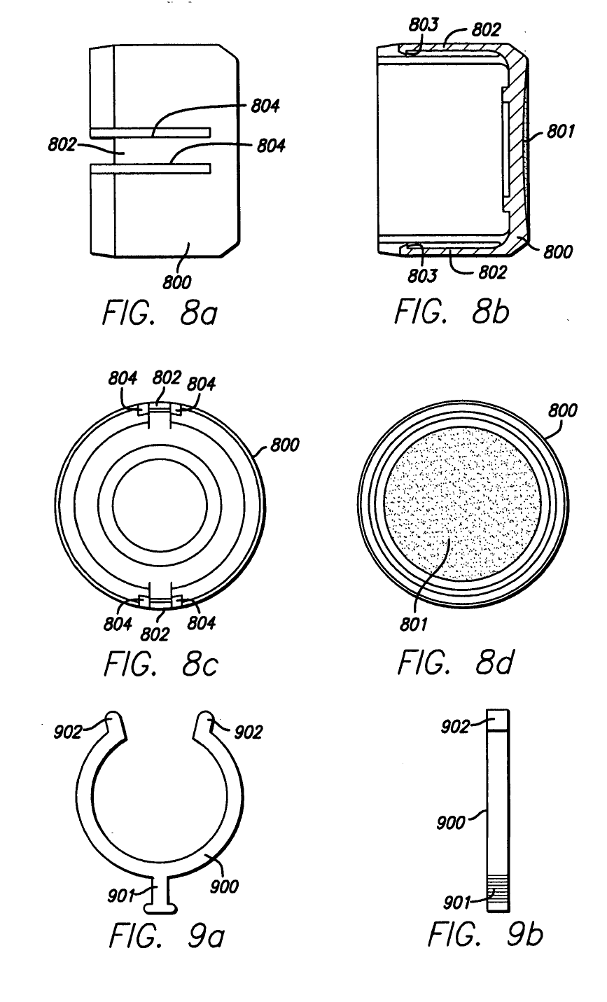

The housing 201 may further be fit with a trigger 800, as depicted in FIG. 8. The trigger 800 is preferably roughly cylindrical, to match the shape of the exterior wall 213 of the housing 201. The distal end of the trigger 800 may have a depression 801 therein, and in preferred embodiments this depression 801 may further be textured for non-slip finger placement during operation of the needle-less injector 100.

The trigger 800 preferably contains at least one retainer hook mechanism 802 used both for securing the trigger 800 to the housing 201 and for mitigating the kickback associated with deploying the compressed gas stored in the engine housing 7000. Without such a safety feature, the force created by release of gas stored in the engine housing 7000 may cause the engine assembly to separate from the remainder of the needle-less injector 100, potentially resulting in, both an improper injection and injury to the user.

The at least one retainer hook mechanism 802 operably mates with the at least one latch retainer mechanism 218 located near the distal end of the housing 201 as the retainer hook 803 at the proximate end of the retainer hook mechanism 802 locks around consecutive saw tooth ridges 219 that preferably comprise the latch retainer mechanism 218. In preferred embodiments, there are two retainer hook mechanisms 802, located opposite one another on the trigger 800, that spatially correspond to two latch retainer mechanisms 218 on the exterior wall 213 of the housing 201.

The at least one retainer hook mechanism 802 and at least one latch retainer mechanism 218 preferably prevent the trigger 800 from rotating about its central axis. In a most preferred embodiment, the sides 804 of the at least one retainer hook mechanism 802 fit around the sides 222 of the at least one latch retainer mechanism 218, preventing such rotation.

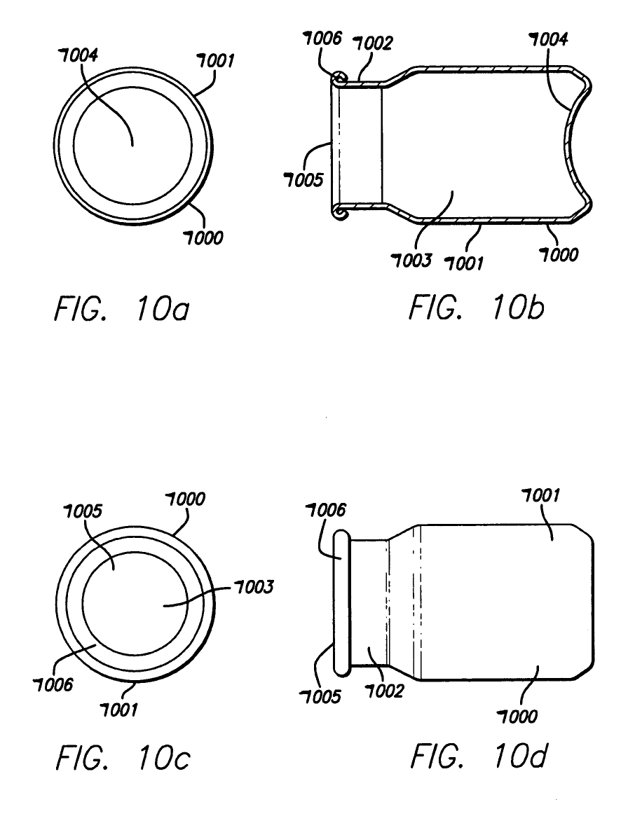

The housing 201 may further be fit with a safety clamp 900, as depicted in FIG. 9. The safety clamp 900 prevents the needle-less injector 100 from being discharged accidentally. The safety clamp 900 is preferably roughly semi-cylindrical in shape to conform to the exterior wall 213 of the housing 201, and resides around the exterior wall 213 of the housing 201 in the clamp indentation 220 that is defined on its proximate end by a ridge 221 and on its distal end by the at least one latch retainer mechanism 218 and the proximate end of the trigger 800 (see FIG. 1b). The safety clamp 900 preferably does not completely encircle the housing 201, but rather encircles only at least half of the housing 201, allowing for easy removal while preventing the clamp 900 from simply falling off of the injector 100. Most preferably, the safety clamp 900 is constructed of a sufficiently elastic material such that temporarily deforming the clamp 900 permits removal thereof from the exterior wall 213 of the housing 201. To aid in this removal, a grip 901 and feet 902 may be included on the safety clamp 900.

The housing 201 is preferably fit with an engine assembly 101, as depicted in FIG. 1b. The engine assembly 101 may further contain an engine housing 7000, as depicted in FIG. 10. The engine housing 7000 is preferably constructed of a material impermeable to a compressed gas stored therein, and has a hollow interior chamber 7003. Most preferably, the engine housing 7000 is comprised of stainless steel or a similar metal. A compressed inert gas is preferably used to drive the needle-less injector 100 and is stored within the engine housing 7000 prior to use. The most preferred gas is carbon dioxide, though other suitable gases may be employed, as well. In most preferred embodiments, the engine assembly 101 is overcharged (i.e., excess compressed gas is stored therein) to allow for use at variable altitudes without hampering the performance characteristics of the needle-less injector 100.

The engine housing 7000 is preferably roughly cylindrical in shape to match the interior wall 212 of the housing 201, though alternate configurations may be utilized. Referring to FIG. 10, the engine housing 7000 may have a portion of wide diameter 7001 and a portion of small diameter 7002, wherein the portion of small diameter 7002 is proximate to the portion of wide diameter 7001. The distal end of the engine housing 7000 may contain a circular depression 7004 and may rest against the trigger 800 (see FIG. 1b). The proximate end of the engine housing 7000 contains an opening 7005, and in preferred embodiments, a closing ridge 7006 encircles the opening 7005.

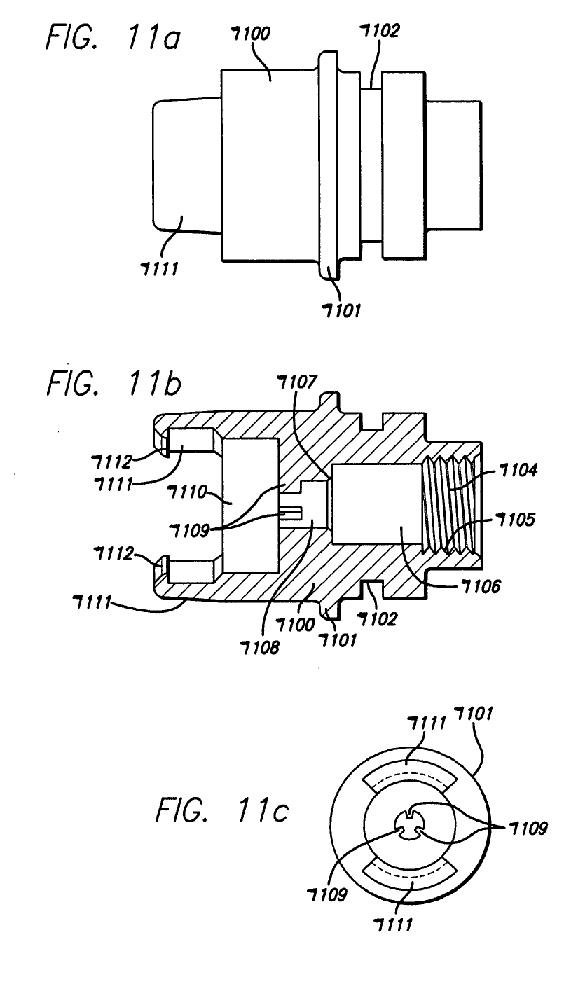

The engine assembly 101 preferably further contains a valve body 7100, as depicted in FIG. 11. The valve body 7100 is preferably roughly cylindrical in its overall shape, and more preferably resides at least partially within the engine housing 7000. The valve body 7100 most preferably has a closing rim 7101 around its outer circumference that rests against the closing ridge 7006 encircling the opening 7005 of the proximate end of the engine housing 7000. Most preferably, a closing ferrule 7200 is wrapped around both the closing rim 7101 and closing ridge 7006 to secure the valve body 7100 and engine housing 7000 to one another (see FIG. 1b).

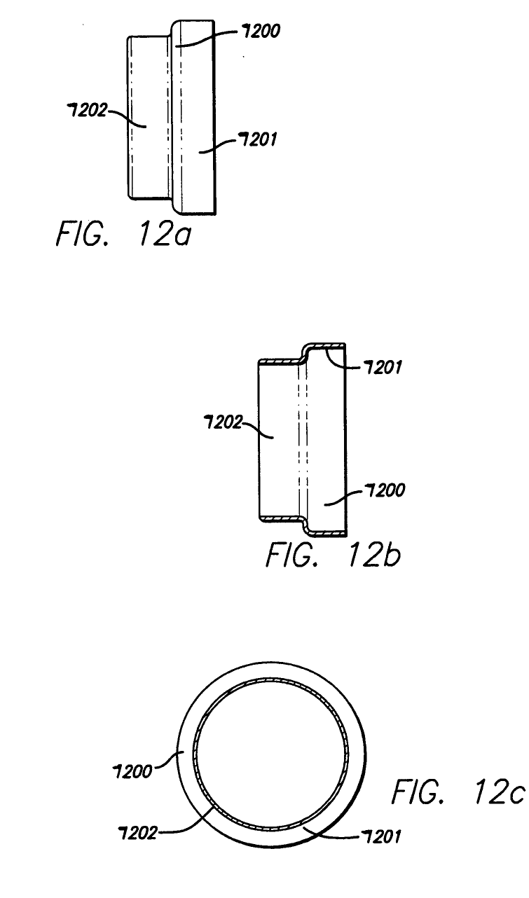

The closing ferrule 7200 is shown in FIG. 12 prior to its distal portion 7201 being mechanically bent around the closing rim 7101 and closing ridge 7006. The proximate portion 7202 of the closing ferrule 7200 is of substantially the same diameter as the exterior of the valve body 7100, such that solely bending the distal portion mechanically couples the valve body 7100 to the engine housing 7000. In FIG. 1, the distal portion 7201 of the closing ferrule 7200 is shown in the bent state. The valve body 7000 preferably has a depression 7102 around its circumference adapted to fit a gasket 7103 (shown in FIG. 1b). The gasket 7103 helps ensure that an airtight seal is maintained between the interior of the engine housing 7000 which contains the gas and the internal atmosphere of the needle-less injector 100.

Referring to FIG. 11, the interior of the valve body 7100 is preferably hollow and comprised of several distinct portions. The distal interior portion 7104 of the valve body 7100 may contain a screw thread engagement 7105, preferably extending from the distal end of the valve body 7100 to the distal end of a first axial cavity 7106. The first axial cavity 7106 may be bounded on its proximate end by a shoulder 7107 that separates this first axial cavity 7106 from a second axial cavity 7108, which is preferably of smaller diameter than the first axial cavity 7106. In preferred embodiments, the shoulder 7107 is an angled edge. Also in preferred embodiments, at least one valve stem guide 7109 protrudes from the wall of the second axial cavity 7108. In a most preferred embodiment, there are at least three such valve stem guides 7109 that serve to substantially prevent the valve stem 7400 from moving in any direction other than along the central axis of the needle-less injector 100 during administration of an injection.

The proximate end of the second axial cavity 7108 preferably terminates at a diffuser-receiving chamber 7110 that is of sufficient diameter such that it encircles a distal end 602 of the diffuser 600. After administration of an injection with the needle-less injector 100, the distal end 602 of the diffuser 600 is most preferably at rest within the diffuser-receiving chamber 7110.

The proximate end of the diffuser-receiving chamber 7110 preferably has at least one grip 7111 extending therefrom. Preferably, the at least one grip 7111 locks around another suitable element of a needle-less injector 100 as the gripping element 7112 is situated on the interior side of the grip 7111. In alternative embodiments, however, the at least one grip 7111 may lock within another element as the gripping element 7112 may be disposed on the exterior side of the grip 7111. In most preferred embodiments, there are two grips 7111 disposed opposite one another each of which contains a gripping element 7112 situated on the interior side of the grip 7111 . In these most preferred embodiments, the two grips 7111 are slid over and lock around the locking ring 605 of the diffuser 600 upon administration of an injection. The combination of a locking ring 605 and grips 7111 assists in mitigating the kickback associated with deploying the compressed gas stored in the engine assembly 101 and ensures that a user fully and properly depresses the trigger 800, since an injection is preferably not deployed until the grips 7111 slip past the locking ring 605.

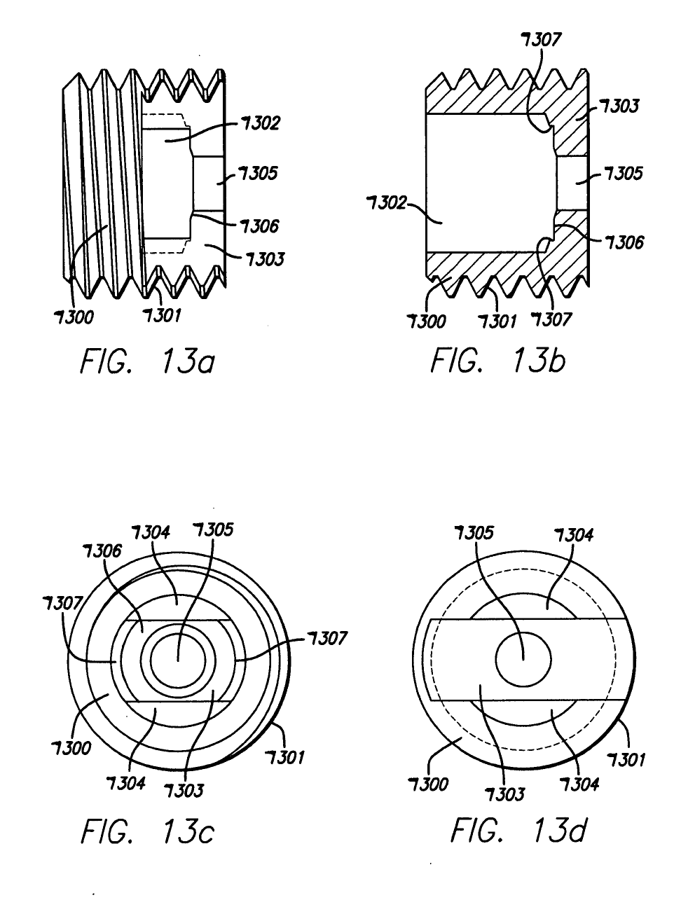

The valve body 7100 preferably further contains a threaded valve guide 7300, as depicted in FIG. 13. The threaded valve guide 7300 is preferably cylindrical in shape and threaded around its exterior wall 7301, such that it may be screwed into the distal interior portion 7104 of the valve body 7100 by interacting with the screw thread engagement 7105. Most preferably, the threading on the exterior wall 7301 of the threaded valve guide 7300 extends along the entirety of the exterior wall 7301 from the distal to the proximate end of the threaded valve guide 7300. The threaded valve guide 7300 may also contain a cylindrical interior cavity 7302 that is unobstructed at the proximate end. The distal end, however, is preferably partially covered with a valve stem guide pane 7303. The valve stem guide pane 7303 preferably provides at least one vent 7304 allowing gaseous communication between the interior cavity 7302 of the threaded valve guide 7300 and the hollow interior chamber 7003 of the engine housing 7000 at the distal end of the threaded valve guide 7300. Also preferably, the valve stem guide pane 7303 includes a hole 7305 at the central axis slightly larger in diameter than the valve stem 7400 that resides therein. Most preferably, the valve stem guide pane 7303 further includes a spring seat 7306 on its proximate surface that is comprised of at least one ridge 7307 that maintains the valve spring 7500 in proper position.

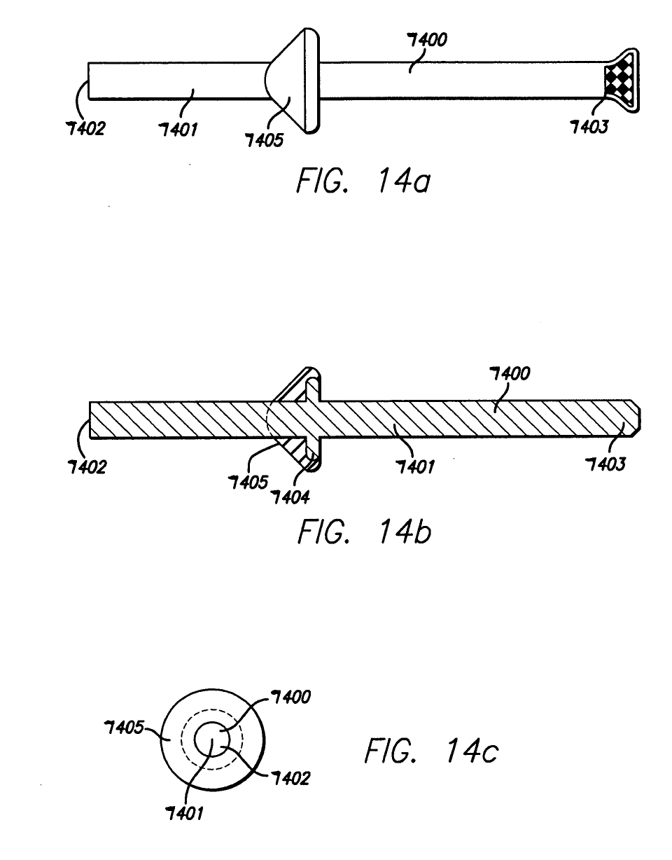

The valve body 7100 preferably further contains a valve stem 7400, as depicted in FIG. 14. The valve stem 7400 is preferably comprised of a substantially cylindrical rod 7401 having a proximate end 7402 which is flat and a distal end 7403 which is preferably pressed or hammer-forged. The distal end 7403 is shown after hammer-forging in FIG. 14a and prior to hammer-forging in FIG. 14b. Most preferably, there is also included a spring ridge 7404 that extends radially from the rod 7401, and a roughly conical valve head 7405 affixed to the proximate and exterior surfaces of the spring ridge 7404 as well as that portion of the rod 7401 immediately proximate to the spring ridge 7404. Most preferably, the valve head 7405 is comprised of a rubber material such as semi-permeable, silicon-based rubber that is sufficiently malleable for use in accordance with the needle-less injector 100. In most preferred embodiments, the angle between the proximate surface of the valve head 7405 and the central axis is substantially similar to the angle of the shoulder 7107 located between the first axial cavity 7106 and second axial cavity 7108 of the valve body 7100.

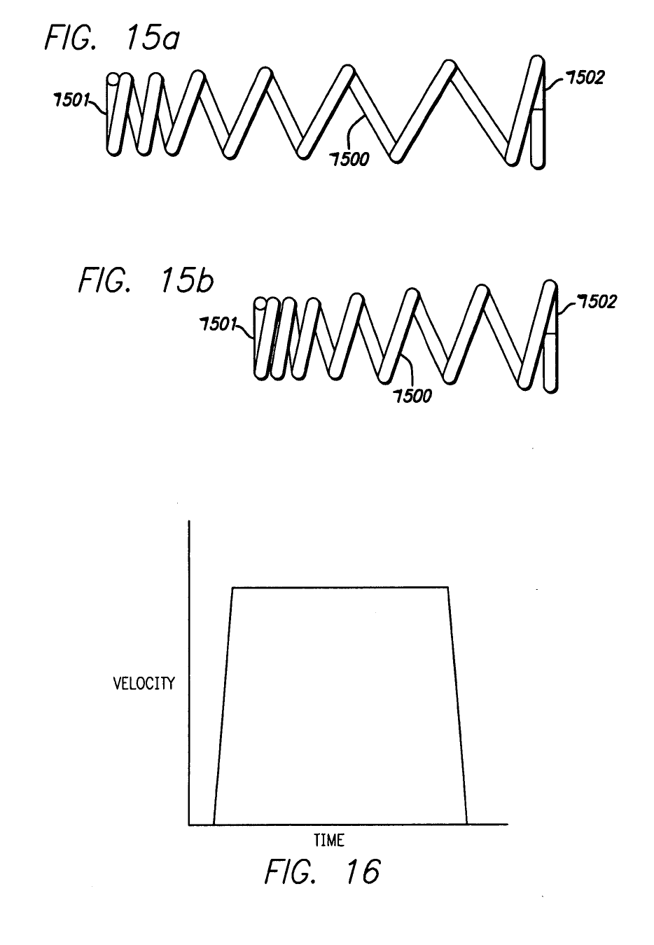

The valve body 7100 may further contain a valve spring 7500, as depicted in FIG. 15. The valve spring 7500 is preferably composed of wire and semi-conical in shape, wherein the proximate end 7501 is smaller in diameter than the distal end 7502. The proximate end 7501 of the valve spring 7500 preferably rests against the distal surface of the spring ridge 7404 on the valve stem 7400, while the distal end 7502 of the valve spring 7500 preferably rests against the proximate surface of the valve stem guide pane 7303 and is held in place radially by the spring seat 7306.

Furthermore, the valve of the instant invention may be repeatedly opened and closed without being destroyed, thus it may be inspected for quality control determinations by opening and closing at least one time prior to the engine assembly 101 being filled with compressed gas. A faulty valve is a concern in any device employing such a mechanism, though it is of particular import in the context of a needle-less injector useful in medical applications, where such a faulty valve may result in the improper dosage of medication.

During the administration of an injection with the needle-less injector, several mechanisms act to mitigate the kickback associated with releasing compressed gas from the engine housing. The grips on the valve body operatively couple with the locking ring on the exterior surface of the diffuser and the retainer hooks on the retainer hook mechanisms operatively lock at each successive saw tooth of the latch retainer mechanisms. Such safety features not only function to avoid potential injury, but further insure proper delivery of degassed fluid through an injection surface.

The above-described modular gas-pressured needle-less injector may be operated as follows. Prior to use, the needle-less injector is assembled with all elements thereof being gamma sterilized with the exception of the engine assembly. The engine assembly is checked for quality control purposes by opening and closing the valve, and thereafter the engine housing is filled with a suitable compressed gas. The interior portion of the housing between the proximate end of the housing and the proximate end of the plunger is then filled with 0.5 ml. of a degassed fluid. The needle-less injector is then assembled and stored, optionally, for a prolonged period of time.

When ready for use (see FIG. 1a), the ampoule cap is removed from the proximate end of the housing by the user. Subsequently, the user also removes the safety clamp by bending and/or distorting the clamp. The user is performing self-administration of an injection and elects to employ the following configuration: the user's index and middle fingers are placed in the arcs of the finger rests for stabilization of the device, with the thumb operably placed against the trigger. The proximate end of the needle-less injector is then positioned roughly perpendicular to the injection surface.

The user then depresses the trigger until the proximate end of the trigger comes to rest against the ridge defining the proximate end of the clamp indentation. During this movement of the trigger, the retainer hook mechanisms and latch retainer mechanisms interact as the retainer hooks lock past consecutive saw teeth that comprise the latch retainer mechanisms.

Forward, axial movement of the trigger causes the engine housing, valve body and threaded valve guide to move, as well. Thus, the grips at the proximate end of the valve body proceed to lock around the locking ring of the diffuser as the distal portion of the diffuser concurrently slides into and partially through the diffuser-receiving cavity of the valve body, coming to rest therein. Simultaneously, the valve stem moves along with the trigger, however, once it comes into mechanical contact with the valve stem support depression in the diffuser it remains stationary relative to the housing. The valve stem and diffuser reach such mechanical contact approximately when the grips slide over and past the locking ring of the diffuser.

When the valve stem and diffuser come into mechanical contact, the valve spring is compressed and the valve opens as the valve head is separated from the shoulder residing between the first and second axial cavities of the valve body. Compressed gas (previously stored in the engine housing, the interior cavity of the threaded valve guide and the first axial cavity of the valve body) may then rush through the gap created between the valve head and the shoulder. The gas rushes through the second axial cavity, past the valve stem guides, through the diffuser-receiving chamber and through the at least one channel in the diffuser. The gas then fills the space defined by the diffuser cup and the expansion cup of the piston, which rest near or against one another prior to gas forcing the two elements apart. The introduction of gas into this space forces the piston in the proximate direction, pushing the plunger through the interior of the housing and correspondingly forcing the degassed liquid from the injector through the at least one orifice in the proximate end of the injector and into and/or through the injection surface. The piston and plunger act in concert as a driver. Once the plunger comes to rest against the proximate end of the housing, excess gas may escape through the exhaust passage in the housing. The user may then dispose of the needle-less injector, the injection having been completed.

### Example 2

### Gas-Powered Needle-Less Injector

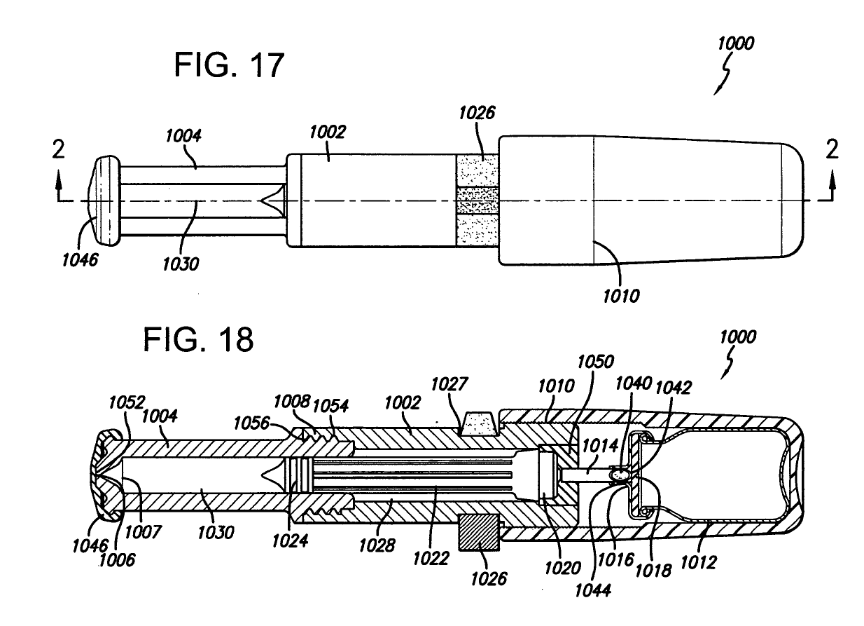

As depicted in FIG. 17, the needle-less injector 1000 may be used as a single dose disposable injector to deliver a dosage of degassed fluid. Precise delivery may be achieved through an orifice with a diameter of approximately 0.0032″ (approximately 0.08 mm). However, larger or smaller diameters, ranging from 0.05 mm to 1.5 mm, may be used, as long as accurate penetration of the skin and delivery of the degassed fluid can be maintained. The degassed fluid is linearly accelerated via pneumatic propulsion. Safety is maintained and inadvertent activation of the needle-less injector 1000 is avoided via a pressure (e.g., resistance) sensitive triggering feature which allows for proper tensioning of the nozzle and orifice at the injection site prior to automatic medication deployment. For example, activation of the needle-less injector 1000 will not occur until the injector is properly positioned to provide the required resistance from the skin surface of the patient to allow for sufficient tension and pressure to be applied to a trigger of the needle-less injector 1000 to activate it to deliver the dosage of degassed fluid. Improper positioning, resulting in insufficient resistance by the skin surface of the patient will prevent the needle-less injector 1000 from being inadvertently activated. For example, tight tolerances between a trigger cap and a housing can prevent the cap from sliding along the housing to trigger the needle-less injector 1000, if the needle-less injector 1000 is more than 10 degrees off of an axis perpendicular to the skin surface of the patient.

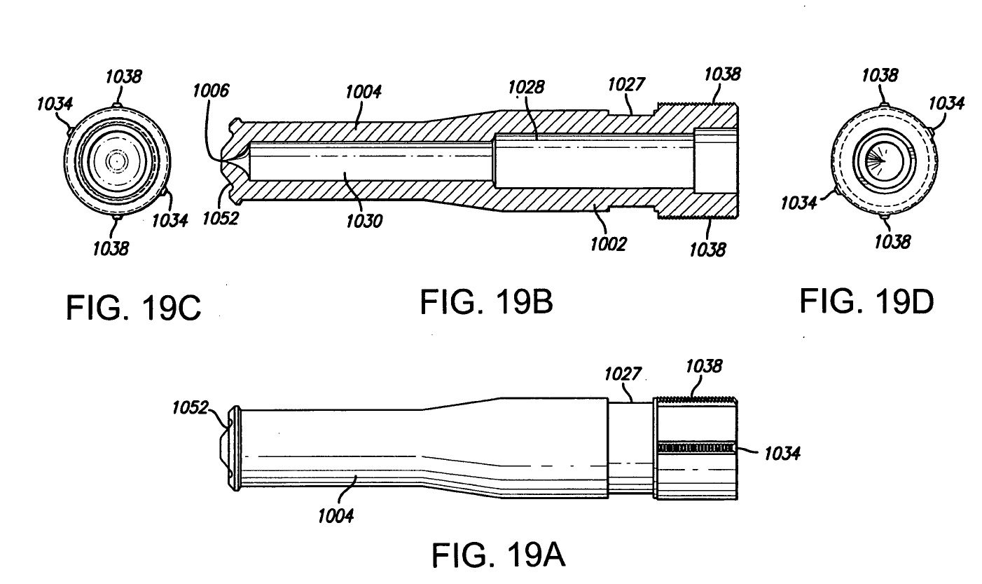

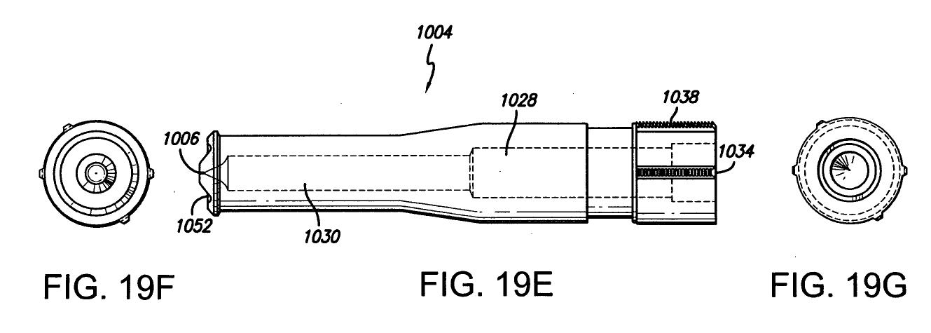

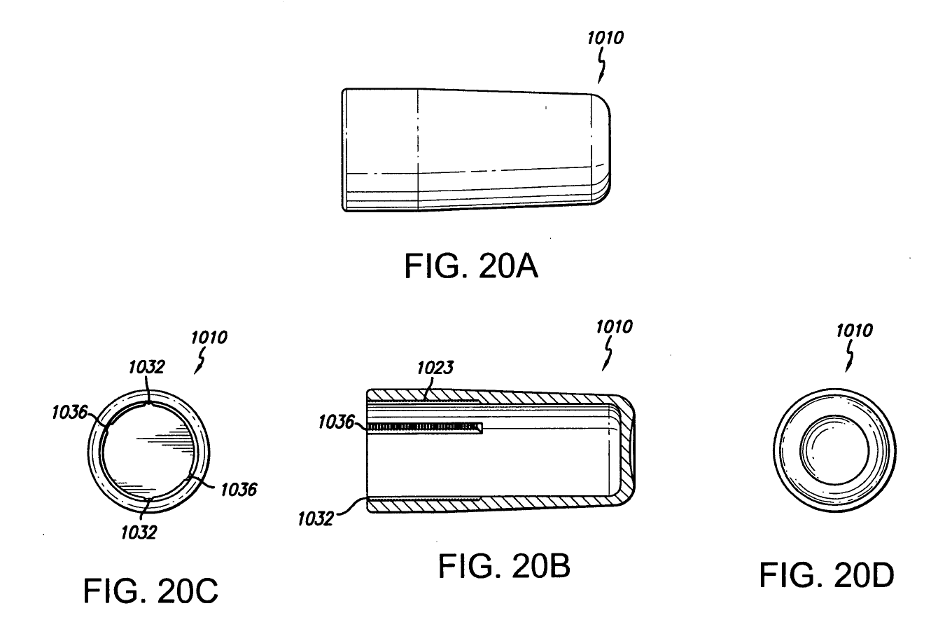

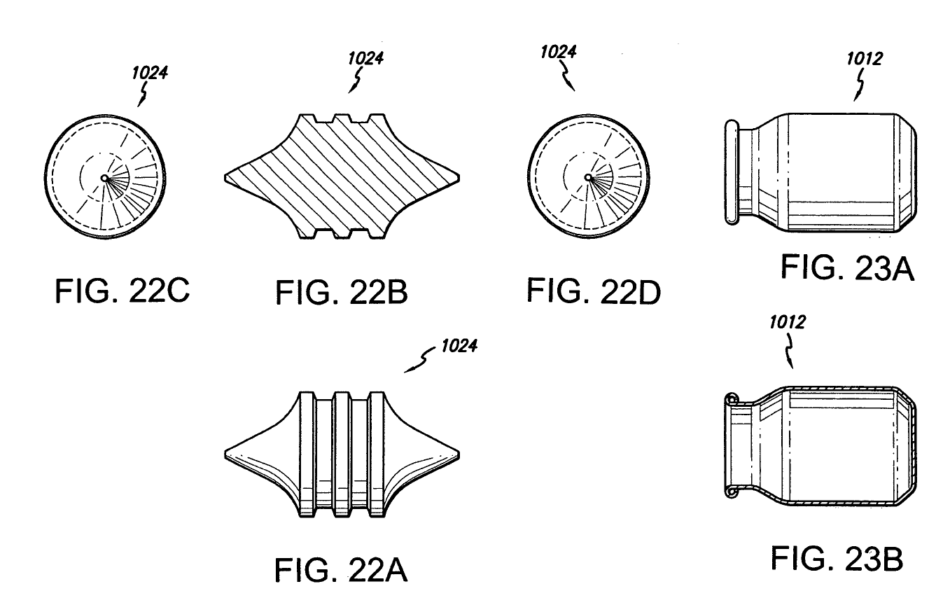

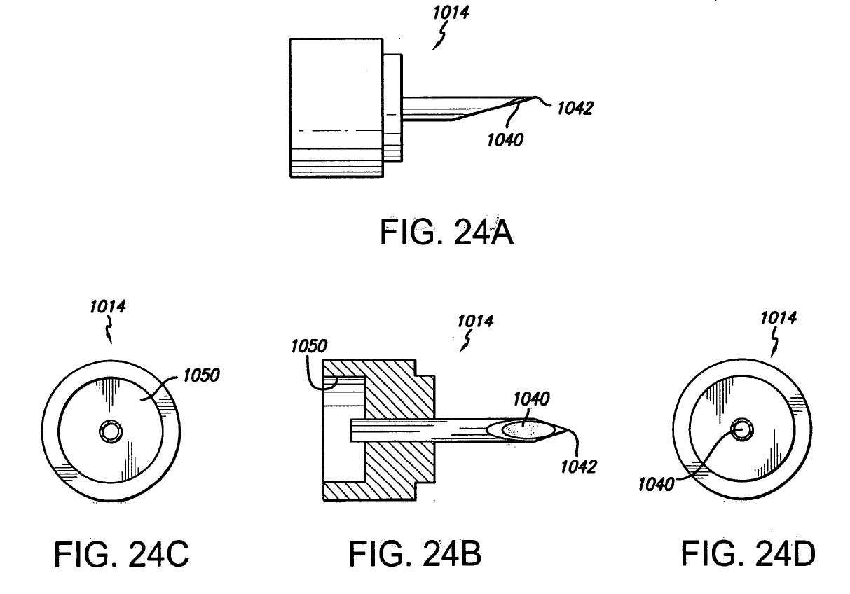

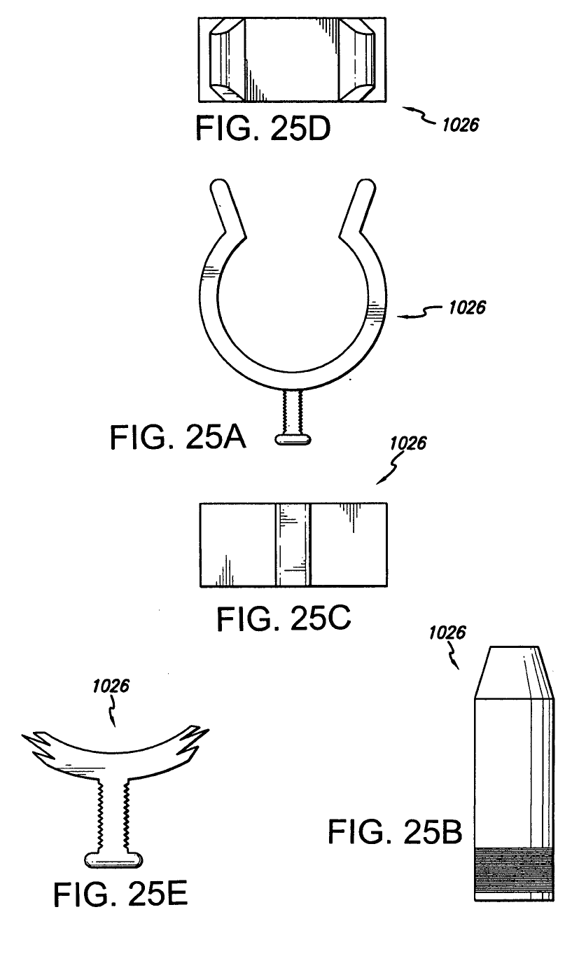

FIGS. 17-25e illustrate a needle-less injector 1000. The needle-less injector 1000 includes a main housing 1002, and an ampoule 1004 having an orifice 1006. The ampoule 1004 includes an open end 1008 that mates with the main housing 1002 through adhesives, welding snap fits, or the like. In alternative embodiments, as shown in FIGS. 19a-19g, the ampoule 1004 is formed as an integral part of the main housing 1002. An actuator cap 1010 mates with the main housing 1002, and a sealed gas charge (or power source) 1012 is contained within the actuator cap 1010. A piercing cannula 1014 is secured to the main housing 1002, and cooperates with a cannula guide 1016 coupled to the gas charge 1012 to guide the piercing cannula 1014 to pierce a diaphragm 1018 that seals in the gas charge 1012. A plunger chamber 1020 works with the other end of the piercing cannula 1014 to assure even distribution of gas pressure when the sealed gas is released from the gas charge 1012. A plunger shaft 1022, that when gas is released, slides within a bore 1028 of the main housing 1002 through the open end 1008 and a bore 1030 of the ampoule 1004 to cause degassed fluid to be expelled through the orifice 1006. A plunger 1024 contained in the ampoule 1004, that fits at the end of the plunger shaft 1022, is moveable by the plunger shaft 1022 and seals the degassed fluid within the ampoule 1004. Thus, the needle-less injector 1000 has an orifice end that includes orifice 1006 and a trigger end that includes the actuator cap 1010. The plunger shaft 1022 is slidably disposed within a bore 1028 of the main housing and the interior bore 1030 of the ampoule 1004.

As the actuator cap 1010 is moved towards the ampoule 1004, the gas charge 1012 is also moved towards the ampoule 1004 and the piercing cannula 1014. The piercing cannula 1014 includes a gas bore (or channel) 1040 formed in the piercing cannula 1014 to act as a conduit to direct the expelled gas into the plunger chamber 1020 to act on the plunger shaft 1022. The piercing cannula 1014 includes a sharp tip 1042 to pierce the diaphragm 1018 of the gas charge 1012. In preferred embodiments, the gas bore 1040 opens up through the sharp tip 1042. However, in alternative embodiments, the sharp tip is solid and includes one or more side ports that provide communication to the gas bore 1040. This design might be desirable if the material forming the diaphragm 1018 of the gas charge 1012 could clog the gas bore 1040. The sharp tip 1042 of the piercing cannula 1014 is contained in a guide bore 1044 formed in the cannula guide 1016 to direct the cannula 1014 to the diaphragm 1018 of the gas charge and to prevent the piercing cannula 1014 from shifting during transport and activation. The other end of the cannula guide 1016 is adapted to attached, by snap fit, threads, detents and slots, adhesives, or the like, to the gas charge 1012.

In preferred embodiments, the diaphragm 1018 is a thin laminate of plastic backed with metal foil that closes off and seals the gas charge 1012. In alternative embodiments, the diaphragm is a frangible metal disk, thin pierceable metal or foil, elastomeric material (such as rubber, plastic or the like), composites, laminates, ceramics, thin glass, or the like. In preferred embodiments, the gas contained in the gas charge 1012 is CO₂. However, alternative embodiments may use other gas, such as air, nitrogen, noble gases, mixtures, liquid/gas combinations, or the like. In a preferred embodiment, the container of the gas charge 1012 is formed from metal. However, other materials, such as plastic, glass, composites, laminates, ceramics, glass, or the like, may be used. In addition, preferred embodiments have a convex bottom as shown in FIGS. 18 and 23c. However, alternative embodiments may use a flat bottom as shown in FIG. 23b or other shapes adapted to mate with the needle-less injector and maintain structural integrity of the gas charge 1012 prior to use.

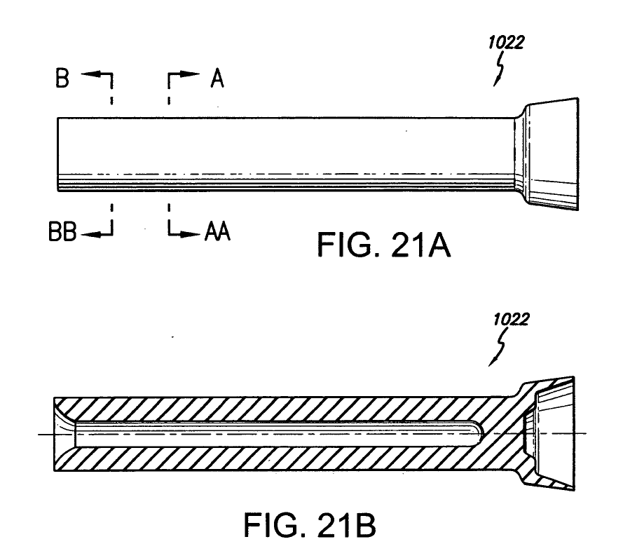

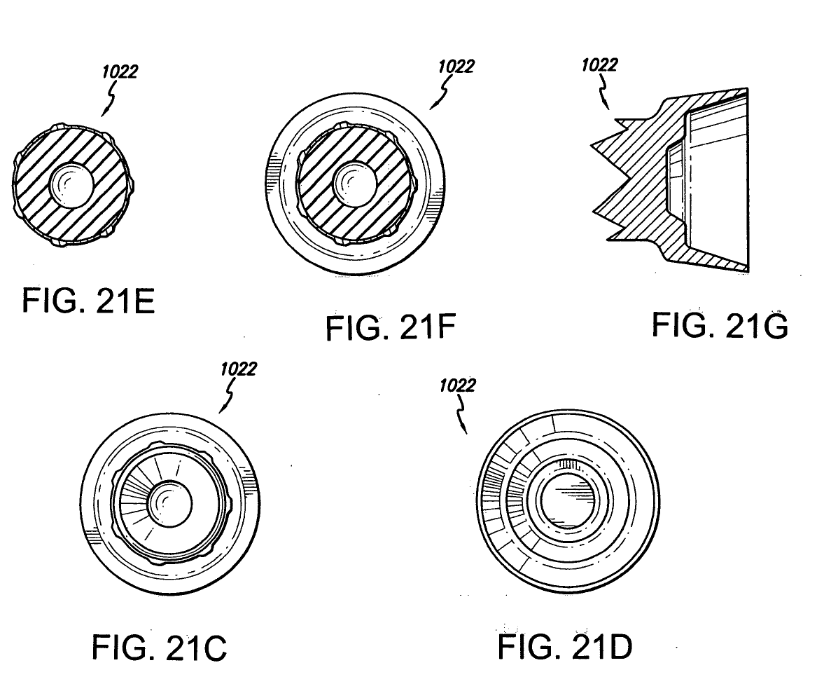

In preferred embodiments, as shown in FIGS. 21a-g, the plunger shaft 1022 has one end inverted cone shaped to receive and seat the corresponding shape of the plunger 1024, and the other end is convex shaped to receive the gas from the gas charge 1012. In alternative embodiments, the front and rear surfaces may be flat, or have other suitable shapes. The plunger shaft 1022 is disposed inside the bore 1028 of the main housing 1022 and the bore 1030 of the ampoule 1004 for sliding movement along their length. In preferred embodiments, one end of the plunger shaft 1022 has substantially the same outer diameter as the inner diameter of the bore 1028 of the main housing 1002 and the other end of the plunger shaft 1022 has substantially the same outer diameter as the inner diameter of the bore 1030 of the ampoule 1004 to provide free sliding movement of the plunger shaft 1022 along the length of the bore 1028 and bore 1030. This also forms an air and fluid tight seal with a minimal friction between the plunger shaft 1022 and the walls of the bore 1028 and 1030.

Preferably, the plunger 1024 is formed of an elastomeric material, such as rubber or plastic, or the like. Also, the plunger 1024 is preferably shaped to fit within a matched recess in the end of the plunger shaft 1022 to minimize twisting or jamming during activation, and matches the shape of the orifice 1006 to minimize leftover degassed fluid at the end of an injection and to maintain the velocity of the escaping degassed fluid throughout the injection. The plunger 1024 has an outer diameter which is substantially the same as the inner diameter of the bore 1030 of the ampoule 1004. The plunger 1024 is disposed between the plunger shaft 1022 and the orifice 1006. The degassed fluid is situated in front of the plunger 1024 (i.e., between the orifice 1006 and the plunger 1024) so that forward movement of the plunger 1024 forces the degassed fluid toward the orifice 1006. The front surface of the plunger 1024 may be configured to match the opening defined by an orifice guide 1007. In preferred embodiments, the front surface of the plunger 1024 has a convex surface to match the concave shape of the orifice guide 1007, whose vertex is the orifice 1006. The shape of the orifice guide 1007 focuses and increases the speed of degassed fluid as it exits the orifice 1006. The matching shapes of the orifice guide 1007 and the plunger 1024 tend to minimize the waste of degassed fluid, since most of the degassed fluid is forced out through the orifice 1006. The shape of the rear surface of the plunger 1024 matches the front surface of the plunger shaft 1022. The similarly shaped configuration provides for an even distribution of the pressure on the rear of the plunger 1024 when the plunger shaft 1022 moves forward. This tends to minimize jams or distortion as the plunger 1024 is driven forward. Preferably, the plunger shaft 1022 and the plunger 1024 are formed as separate pieces. However, in alternative embodiments, the plunger shaft 1022 and the plunger 1024 are formed as an integrated piece either by attaching the plunger 1024 to the plunger shaft 1022 or by molding the plunger shaft 1022 to include the plunger 1024.

To use the needle less injector 1000, the user removes the protective cap 1046 that may cover the orifice 1006 of the ampoule 1004. The user also removes the safety clip 1026, where included. Next, the user places the orifice 1006 and end of the ampoule 1004 against the tissue (such as skin, organs, different skin layers or the like) so that the needle-less injector 1000 is generally perpendicular to the tissue, as described above. The user then presses on the actuator cap 1010 to move it towards the ampoule 1004. The actuator cap 1010 moves after a predetermined force threshold is reached and the tissue resists further forward movement of the injector 1000. As the actuator cap 1010 moves towards the ampoule 1004, the gas charge 1012 and cannula guide 1016 are moved towards the sharp tip 1042 of the piercing cannula 1014, which eventually pierces the diaphragm 1018 to release the gas in the gas charge 1012. The gas then flows down the gas bore 1040 in the piercing cannula 1014 filling the plunger chamber 1020, and then presses on the plunger shaft 1022. As the released gas escapes, the pressure quickly increases to drive the plunger shaft 1022 forward, which in turn drives the plunger 1024 forward towards the orifice 1006 in the ampoule 1004. As the plunger 1024 travels forward, degassed fluid is expelled out of the orifice 1006 to pierce the tissue and deliver the degassed fluid below the surface of the tissue.

In preferred embodiments, the open end 1008 of the ampoule 1004 has threads 1054 on the outer diameter and matching threads 1056 are formed inside of the main housing 1002 to screw-in the ampoule 1004. Although not shown in the drawings, an O-ring may be placed between the ampoule 1004 and the main housing 1002 to provide an additional air and fluid tight seal. Using separate parts provides the advantage of being able to assemble the needle-less injector 1000 when needed or just prior to giving an injection. Also, the needle-less injector 1000 can be disassembled as desired. This assembly option allows the user to select a variety of different degassed fluids or dosages while minimizing the number of complete needle-less injectors 1000 that must be carried or stocked. In addition, a user may store the ampoule 1004 in different environments, such as a refrigerator for perishable degassed fluids, and minimize the refrigerated storage space, since the rest of the needle-less injector 1000 does not require refrigeration. It also facilitates manufacture of the needle-less injector 1000, since the needle-less injector 1000 and the ampoule 1004 may be manufactured at different times. Alternatively, as shown in FIGS. 19a-19g, the ampoule 1004 is formed as an integral part of the main housing 1002. This reduces the number of molded parts and the overall cost of the injector device 1000.

### Example 3

### Needle-Less Injector Including a Latch

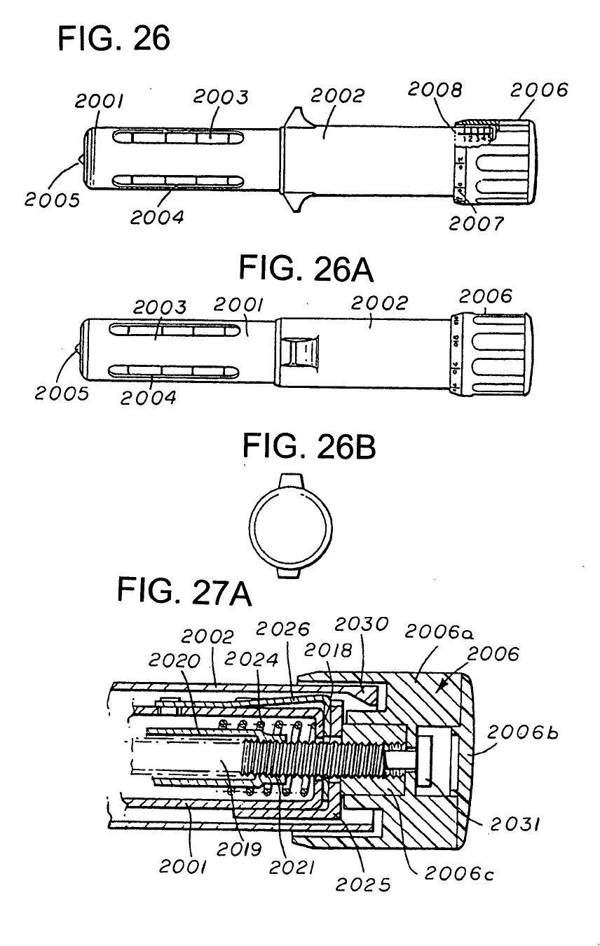

As depicted in FIG. 26, a needle-less injector comprises a tubular body 2001, which retains a cartridge 2003 pre-filled with a degassed fluid, and visible through one or more windows 2004 in the body 2001. The body 2001 has an aperture in the end to permit a nozzle 2005 to protrude. A finger nut 2006 is used by the operator to control the dose volume, and has markings 2007 thereon to indicate its position relative to a scale 2008 on sliding sleeve 2002, which is arranged co-axially on the body 2001.

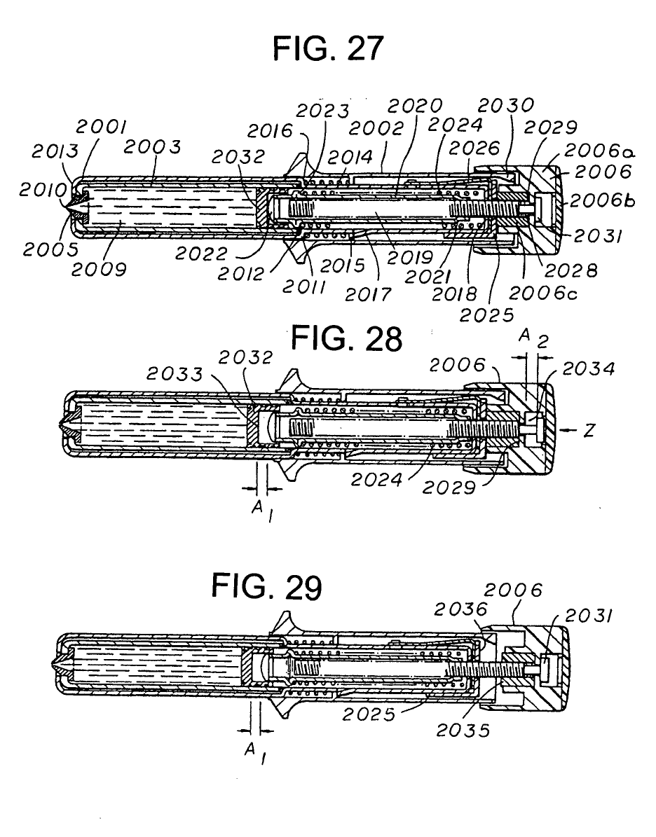

In FIG. 27, the cartridge 2003 is shown filled with degassed fluid 2009, and fitted with a nozzle 2005 having an orifice 2010, and a free piston 2032. The nozzle 2005 may be a separate component as shown, sealingly fixed into the cartridge 2003, or may be formed integrally with the cartridge 2003. Preferably the cartridge 2003 is made of a transparent material compatible with the degassed fluid 2009, to enable the contents to be viewed through the windows 2004 in body 2001. The cartridge 2003 abuts a shoulder 2011 formed on body 2001, and is retained in this position by the crimped end 2013 of body 2001. The cartridge 2003 is biased toward the crimped end 2013 by a resilient gasket or wave washer 2012 interposed between shoulder 2011 and an end face of the cartridge 2003.

The sliding sleeve 2002 is assembled co-axially on body 2001 and is urged away from nozzle 2005 by a spring 2014 supported by a shoulder 2016 on body 2001 and acting on a shoulder 2015. The extent of the rearward movement is limited by shoulder 2015 resting on one or more stops 2017. A cam 2030 is formed inside the sleeve, so that when the sleeve is moved towards the nozzle 2005, the cam strikes a latch 2026 to initiate the injection.

Support flange 2018 is formed on the end of the body 2001 and has a hole co-axially therein through which passes a threaded rod 2019, which may be hollow to save weight. A tubular member 2020 is located coaxially within the rear portion of the body 2001 and has an internal thread 2021 at one end into which the rod 2019 is screwed. The other end of the tubular member 2020 has a button having a convex face 2022 pressed therein. Alternatively, the tubular member 2020 may be formed to provide a convex face 2022. A flange 2023 is formed on the tubular member, and serves to support a spring 2024, the other end of which abuts the inside face of support flange 2018. In the position shown, the spring 2024 is in full compression, and held thus by the nut 2006 which is screwed onto threaded rod 2019, and rests against the face of the bridge 2025. In the illustrated embodiment the nut 2006 consists of three components, held fast with one another, namely a body 2006a, an end cap 2006b and a threaded insert 2006c. The insert 2006c is the component which is screwed on to the rod 2019, and is preferably made of metal, for example brass. The other components of the nut can be of plastics materials.

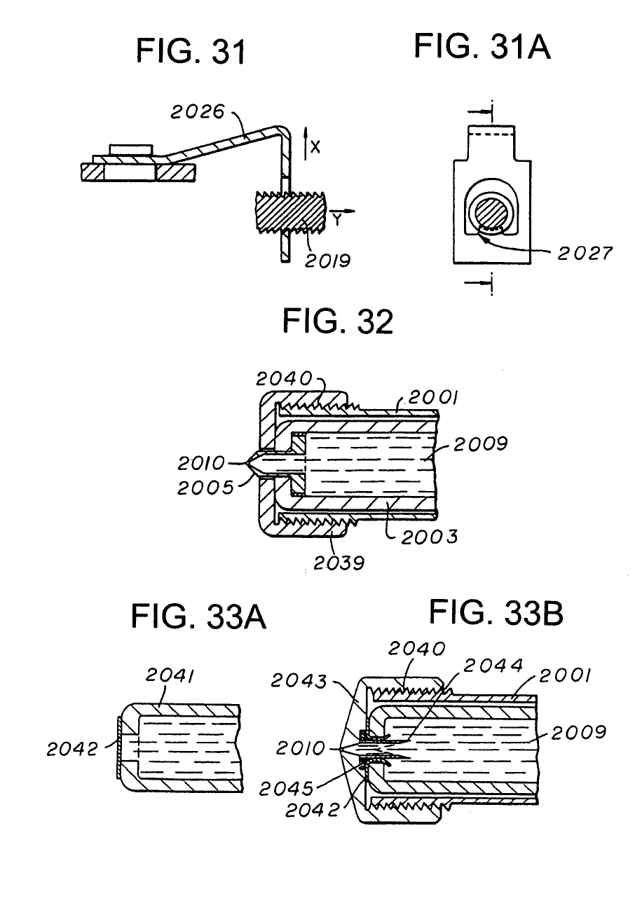

Beneath the bridge and guided by same is a latch 2026 which is attached to the body 2001 and resiliently engaged with one or more threads on the screwed rod 2019. The latch 2026 is shown in more detail in FIG. 31, and is made from a spring material and has a projection 2027 which has a partial thread form thereon, so that it engages fully with the thread formed on rod 2019. The latch 2026 is attached to body 2001 and has a resilient bias in the direction of arrow X, thus maintaining its engagement with the thread on rod 2019. Movement against the direction of arrow X disengages the latch from the thread. As will be described, the rod 2019 will be translated without rotation in the direction of arrow Y when setting the impact gap, and the latch 2026 will act as a ratchet pawl. The thread on rod 2019 is preferably of a buttress form (each thread has one face which is perpendicular or substantially perpendicular, say at 5°, to the axis of the rod, and the other face is at a much shallower angle, say 45°), giving maximum strength as a latch member, and a light action as a ratchet member.

Referring again to FIG. 27, nut 2006 is screwed part way onto threaded rod 2019, so that there is a portion of free thread 2028 remaining in the nut 2006, defined by the end of rod 2019 and stop face 2029 in nut 2006. A stop pin 2031 has a head which bears against the stop face 2029, and a shaft which is fixedly secured to the inside of rod 2019, for example by adhesive. The stop pin 2031 prevents the nut 2006 being completely unscrewed from rod 2019, since when the nut 2006 is rotated anticlockwise, it will unscrew from the rod 2019 only until the head of pin 2031 contacts the face of the recess in the nut 2006 in which it is located. The pin 2031 also defines the maximum length of free thread in nut 2006 when fully unscrewed.

Referring to FIG. 28, the first stage in the operating cycle is to rotate the nut 2006 on threaded rod 2019 in a clockwise direction (assuming right-hand threads, and viewing in direction arrow Z). The rod 2019 is prevented from turning, since the friction between the screw thread and the latch 2026 is much higher than that between the nut 2006 and the rod 2019. This is mainly because the nut is unloaded, whereas the rod 2019 has the full spring load engaging it with the latch 2026. The rod 2019 therefore moves into the nut 2006 as far as the stop face 2029. Alternative ways could be used to prevent the rod 2019 from turning, for example using a ratchet or the like, or a manually operated detent pin. Since the threaded rod is attached to the tubular member 2020, by the interengagement of the thread on rod 2019 with the thread 2021 on member 2020, the latter is also moved rearwards (i.e. to the right as viewed in FIG. 27), increasing the compression on spring 2024, and thus creates a gap A₁ between the convex face 2022 of the tubular member 2020 and the inner face 2033 of piston 2032. When the rod 2019 is fully screwed into nut 2006 the stop pin 2031 projects a distance A₂ from face 2034 which is equal to the gap A₁.

Referring to FIG. 29, nut 2006 is now rotated anticlockwise until it contacts stop pin 2031, which locks the nut 2006 to the threaded rod 2019. There is now a gap between face 2035 on nut 2006 and the abutment face 2036, which gap is equal to gap A₁. Continued rotation of the nut now rotates the threaded rod also, because of the attachment of the shaft of the pin 2031 to the side of the rod 2019, and unscrews it in a rearward direction. The face 2035 on nut 2006 thus moves further away from its abutment face 2036 on bridge 2025. The increase in the gap is equivalent to the required stroke of the piston, and thus the total gap is the sum of the impact gap A₁ and the required stroke. The nut 2006 has markings on the perimeter which are set to a scale on the sliding sleeve 2002, in the manner of a micrometer. The zero stroke indication refers to the position of nut 2006 when it first locks to the threaded rod 2019, and immediately before the threaded rod is rotated to set the stroke.

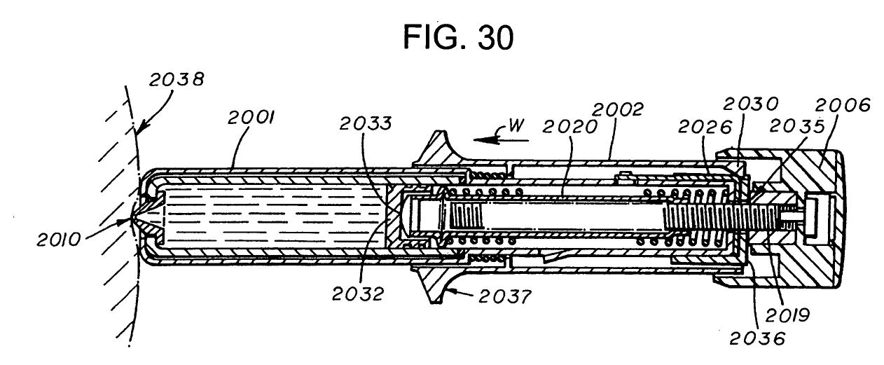

The needle-less injector is now ready to inject the degassed fluid, and referring to FIG. 30, the needle-less injector is held in the hand by sliding sleeve 2002, and the orifice 2010 is placed on the epidermis 2038 of the subject. Force is applied on the finger stops 2037 in the direction of arrow W. The sliding sleeve 2002 compresses spring 2015 and moves towards the subject so that the force is transmitted through spring 2014 to the body 2001 and thus to the orifice 2010, so as to effect a seal between the orifice 2010 and epidermis 2038. When the contact force has reached the predetermined level, the cam 2030 on sliding sleeve 2002 contacts latch 2026 and disengages it from threaded rod 2019. The spring 2025 accelerates the tubular member 2020 towards the piston through the distance A₁, and the convex face 2022 strikes the face 2033 of piston 2032 with a considerable impact. The tubular member 2020 thus acts as an impact member or ram. Thereafter the spring 2024 continues to move the piston 2032 forward until the face 2035 on nut 2006 meets the face 2036 on bridge 2025. The impact on the piston causes within the degassed fluid a very rapid pressure rise—effectively a shock wave—which appears almost simultaneously at the injection orifice, and easily punctures the epidermis. The follow-through discharge of the degassed fluid is at a pressure which is relatively low but sufficient to keep open the hole in the epidermis.

Spring 2024 should be given sufficient pre-compression to ensure reliable injections throughout the full stroke of the ram. A 30% fall in force as the spring expands has been found to give reliable results. Alternatively, a series stack of Belleville spring washers in place of a conventional helical coil spring can give substantially constant force, although the mass and cost will be slightly higher.

The embodiment thus described provides an inexpensive, compact, convenient and easy-to-use disposable needle-less injector, capable of making sequential injections from a single cartridge of medicament. The power source is a spring which is pre-loaded by the manufacturer, and the cartridge is also pre-filled and assembled into the needle-less injector. Thus the user merely rotates the single adjustment nut and presses the injector onto the epidermis, and the injection is triggered automatically. The size and mass of the needle-less injector will depend on the quantity of degassed fluid contained therein, but typically, using a lightweight aluminum body and thin-walled construction where possible, a 5 ml needle-less injector would be about 135 mm long, 24 mm diameter (nut), with a mass of about 85 g including degassed fluid.

### Example 4

### Single-Use Needle-Less Injector Including a Latch and Two-Component Injectate

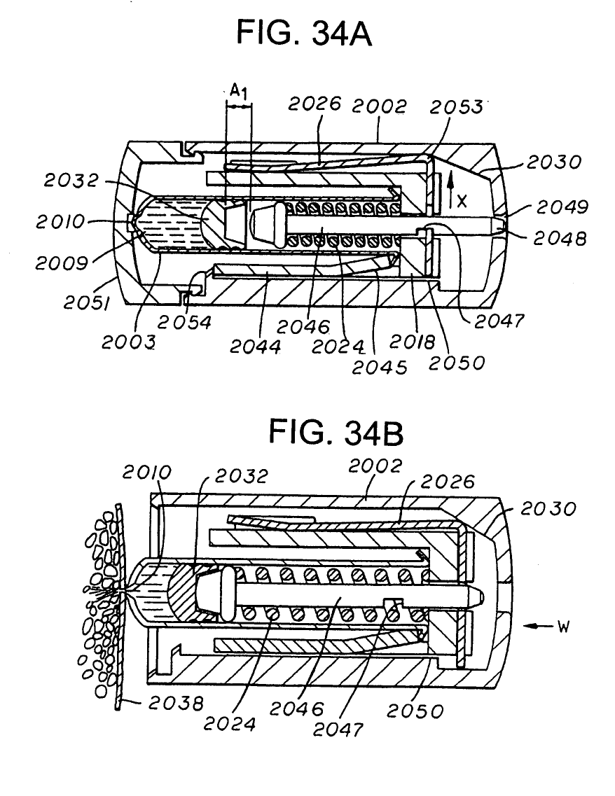

The embodiment shown in FIGS. 34a and 34b is a single use disposable needle-less injector. Referring to FIG. 34a, cartridge 2003 containing degassed fluid 2009 and free piston 2032 is firmly located in the injector casing 2044 and retained by one or more resilient lugs 2045, so that there is no longitudinal free play. A ram 2046 is located concentrically with the cartridge and such that there is an impact gap A₁ between the adjacent faces of the piston 2032 and ram 2046. Ram 2046 is urged towards piston 2032 by spring 2024, but is prevented from moving by latch 2026 supported on flange 2018 and engaged with notch 2047 in the stem of the ram 2046. Latch 2026 is made from a resilient material, and is configured to apply a bias in the direction of arrow X. A sliding sleeve 2002 is located over the casing 2044, with cam surface 2030 just touching the bend 2053 on latch 2026, and retained on casing 2044 by lug 2054. Thus the latch 2026 acts also as a spring to bias the sleeve 2002 in direction of arrow X relative to the casing 2044. The degassed fluid 2009 and orifice 2010 are protected by a cap 2051 snap fitted to the sliding sleeve 2002 as shown, or attached to the cartridge 2003. Distal end 2048 of ram 2046 is located within aperture 2049 in sliding sleeve 2002, giving visual and tactile indication that the injector is loaded and ready for use.

Referring now to FIG. 34b, to make an injection, cap 2051 is removed and the orifice 2010 is placed on the subject's skin 2038, with the axis of the injector approximately normal to the skin. Sufficient force is applied on sliding sleeve 2002 in the direction of arrow W to overcome the biasing force of the latch 2026 on cam surface 2030. The sleeve 2002 moves in the direction of arrow W and the cam surface 2030 thus disengages the latch 2026 from the notch 2047 in ram 2046 which is then rapidly accelerated by spring 2024 to strike piston 2032, and the injection is accomplished as previously described. The point at which the latch 2026 disengages from the ram 2046 is directly related to the reaction force on the subject's skin, and by suitable selection of components, accurate and repeatable placement conditions may be met, ensuring predictable triggering of the injection. A safety bar 2050 on sliding sleeve 2002 prevents accidental disengagement of the latch 2026 (by dropping, for example), and this safety feature may be augmented by a manually operated detent (not shown) that prevents movement of the sliding sleeve 2002 until operated. In an alternative arrangement (not shown) the latch 2026 may be biased in the opposite direction to that described, so that it tries to disengage itself from the notch 2047 but is prevented from doing so by a bar on sliding sleeve 2002. Movement of the sliding sleeve 2002 and bar permits the latch 2026 to disengage itself from the notch 2047, thus initiating the injection: in this example a separate spring means may be required to bias the sliding sleeve 2002 against the direction of arrow W.

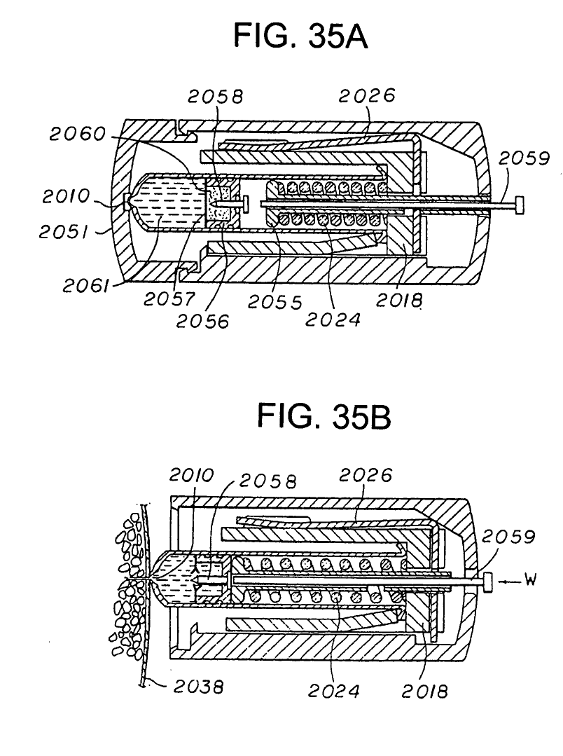

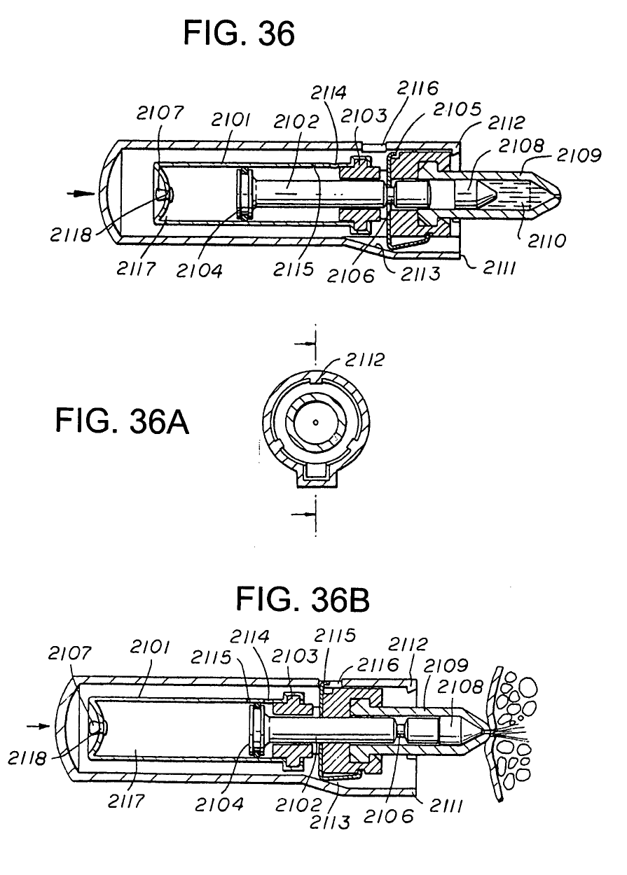

The embodiment shown in FIGS. 35a and 35b is similar to that shown in FIGS. 34a and 34b and described above, but modified to permit the storage of a lyophilized drug and degassed fluid, or other two-part formulations including a degassed fluid. FIG. 35a shows a single dose needle-less injector, loaded and ready for use. Free piston 2056 is hollow and stores one component 2060 of the medicament—for example a lyophilized drug—which is retained in piston 2056 by frangible membrane 2057 which also separates the drug 2060 from a degassed fluid 2061 stored in cartridge 2003. A membrane cutter 2058, which has one or more cutting edge, is sealingly and slidingly located in piston 2056, so that its cutting edge is a small distance from the frangible membrane 2057. Ram 2055 is hollow, and located within its bore is a cutter operating rod 2059. Referring also to FIG. 35b, the rod 2059 is pushed in the direction of arrow W so that it acts on membrane cutter 2058. The membrane cutter 2058 cuts membrane 2057, thus allowing the degassed fluid 2061 to mix with and dissolve the drug 2060. The needle-less injector may be agitated to accelerate the mixing process. Throughout the membrane cutting and mixing period, protective cap 2051 seals orifice 2010 to prevent loss of degassed fluid 2061 and/or the mixture thereof with the lyophilized drug or other medicament 2060. After sufficient time has elapsed to ensure thorough dissolution of the drug, cap 2051 is removed, orifice 2010 is placed on the subject's skin, and the injection is accomplished as previously described.

Except during the injection, the main reaction forces of the spring 2024 and the latch 2026 are taken on the support flange 2018. During the injection, although the shock forces are high, they are of very short duration, and therefore the body components may be of very lightweight construction. Thus, although the use of thin metal tube is described in the embodiments, plastics may be used for most structural parts because they would not be subject to sustained forces which could lead to creep and distortion.

Whilst the shape of the nozzle may be such to achieve optimum sealing efficiency and comfort, the geometry of the orifice within the nozzle should have a length to diameter (L:D) ratio of preferably not more than 2:1, preferably in the order of 1:2, and the exit of the orifice should be placed directly onto the epidermis. It is sometimes necessary to use multiple orifice nozzles, particularly when dispensing large volumes, and each orifice in the nozzle should ideally have a maximum L:D ratio of 2:1, preferably 1:2.

### Example 5

### Electric-Powered Needle-Less Injector

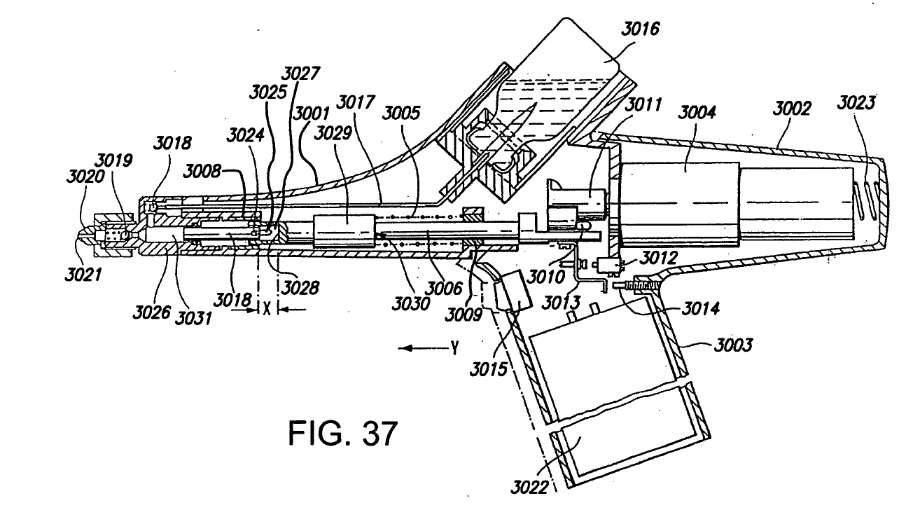

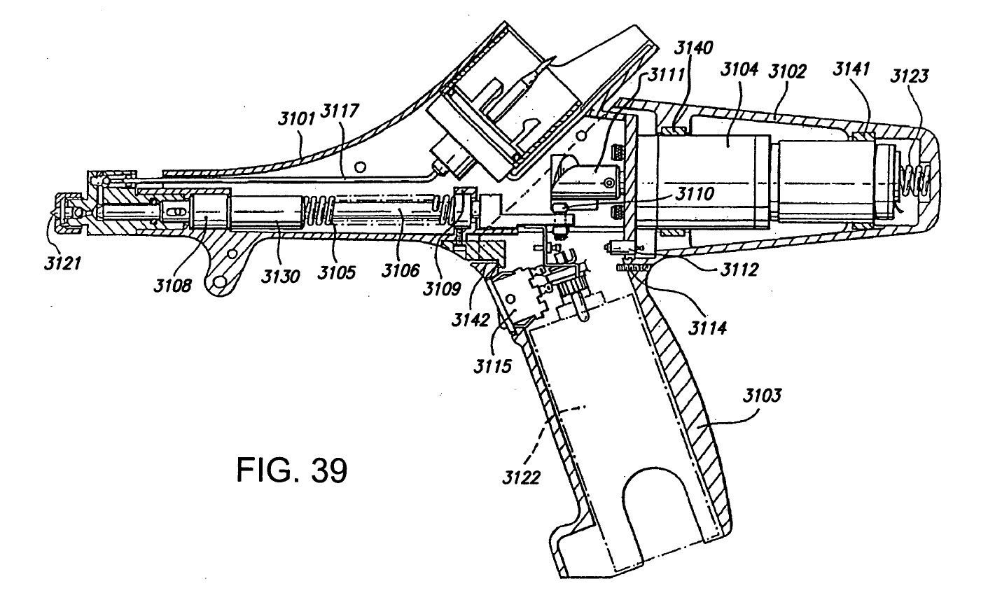

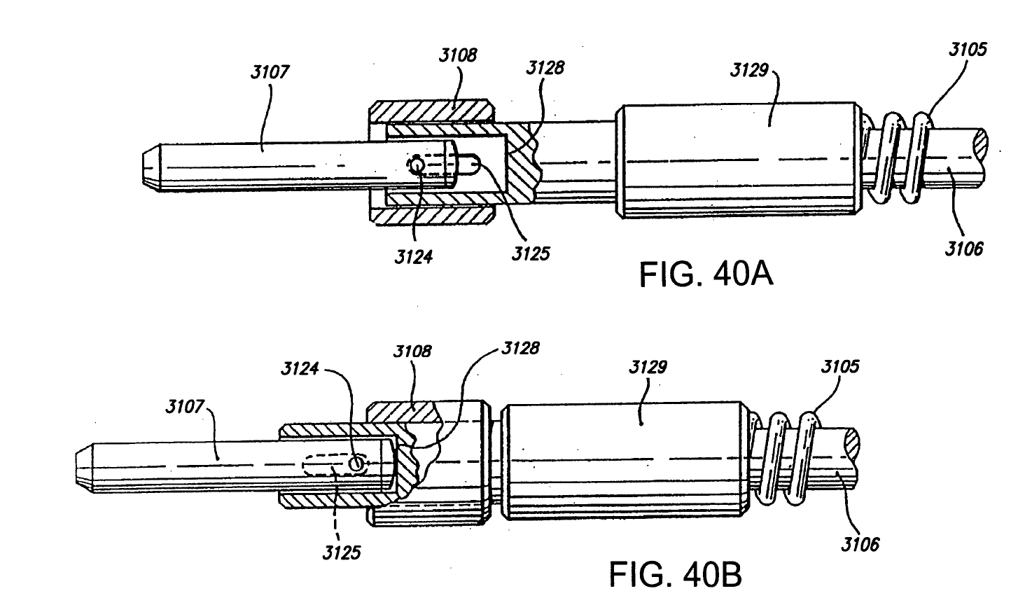

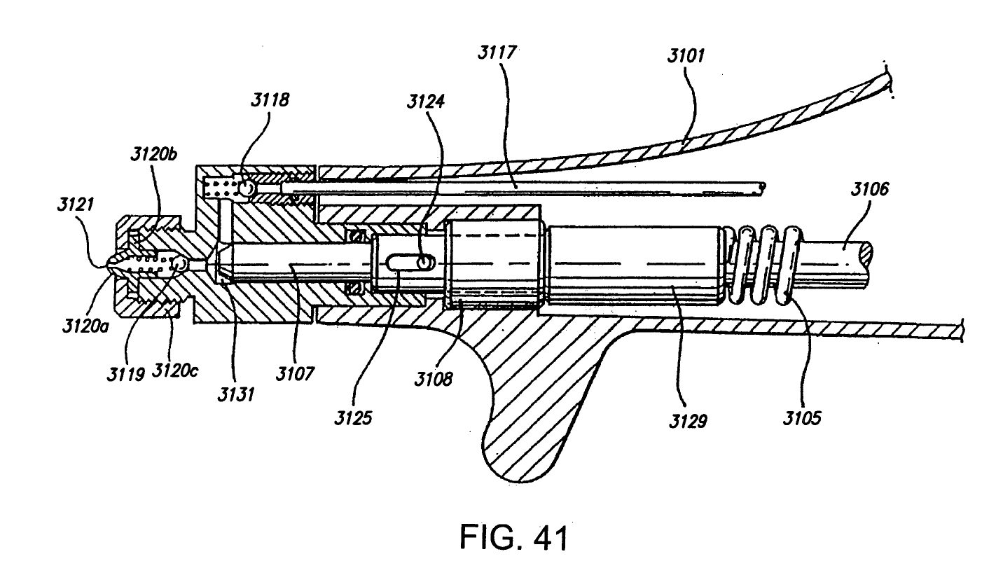

The needle-less injector shown in FIG. 37, comprises an outer casing having a front section 3001 and a rear section 3002. Section 3002 may be displaced along the longitudinal axis of the injector relative to section 3001, from which it is urged apart by a spring 3023. The sections are held together against the force of the spring by a restraining block which is not shown in FIG. 37 but which is of similar form to the block shown in FIG. 39 in relation to a second embodiment. The front end of section 3001 supports a cylinder 3026 in which a piston 3007 is sealingly located. The piston 3007 is preferably hollow, but closed at both ends, in the case of the righthand end by a hard cap. The cylinder 3026 is connected via a non-return valve 3018, biased to its closed position by a compression spring, and a tube 3017 to a reservoir 3016 containing a degassed fluid to be injected. The reservoir has an air inlet (not shown) to permit air to enter the bottle as the degassed fluid is dispensed therefrom. A discharge nozzle 3020 is sealingly connected to the cylinder 3026, and a non-return valve 3019, biased to its closed position by a compression spring, prevents air being drawn into the cylinder during the induction stroke.

The piston 3007 is loosely located within a hole 3027 in the end of a connecting rod 3006, so that it may move freely in a longitudinal direction. A pair of pins 3024 is fixed to the piston 3007, the pins extending radially therefrom on opposite sides thereof. Each pin slides in a slot 3025 in the connecting rod 3006. In the extreme leftward position of the piston 3007, the pins 3024 are at the lefthand ends of their respective slots. However, in the extreme righthand position of the piston 3007 the pins do not reach the righthand ends of their respective slots. That position is defined by a face 3028 at the end of hole 3027, the righthand end of the piston 3007 meeting that face before the pins can reach the righthand end of their slots. The connecting rod 3006 is slidingly located in bearings 3008 and 3009, and urged in the forward direction by a compression spring 3005 one end of which acts on a face 3030 of a mass 3029 which is integral with the connecting rod 3006. A distinct mass 3029 which is identifiable as such is not always necessary for example if the mass of the rod 3006 itself is sufficient. The other end of the spring 3005 reacts against the end face of the bearing 3009.

A motor-gearbox assembly 3004 is housed in casing section 3002 but attached to front section 3001 and the output shaft carries a cylindrical cam 3011 to which is engaged a follower 3010 attached to the connecting rod 3006. The motor is described below as being electric, but could be of some other type, for example gas powered. A resilient microswitch trip 3013 is mounted on the connecting rod 3006, so that when the connecting rod 3006 is retracted against the spring 3005 (by rotation of the cam 3011), at a predetermined position, the trip 3013 operates a normally closed microswitch 3012 attached to the front section 3001. The rear section 3002 has a handle part 3003 which houses an electrical battery 3022 and a trigger switch 3015. The battery is connected in series with the trigger switch 3015, the microswitch 3012 and the motor 3004.

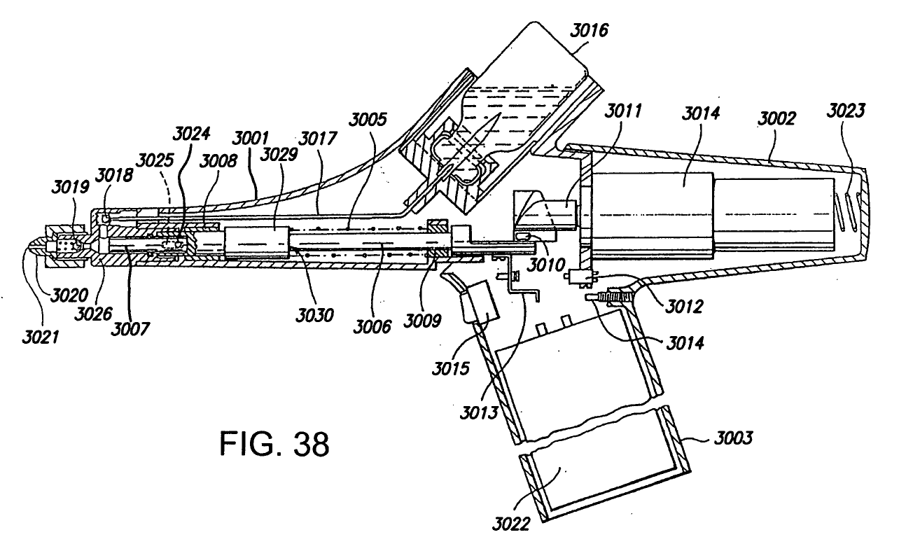

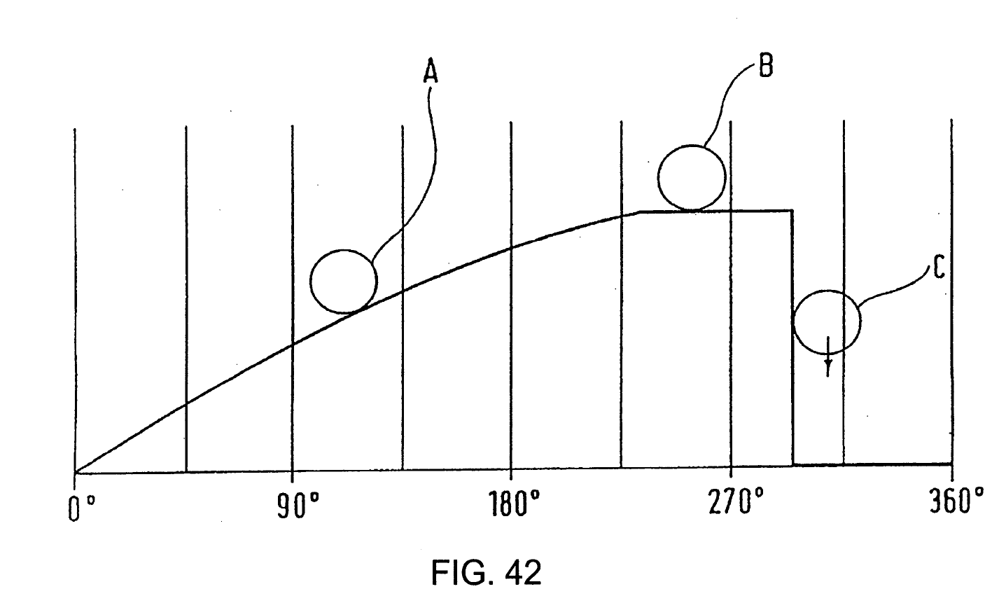

Referring to FIG. 38 (which shows the needle-less injector in the discharged condition) the trigger switch 3015 is operated, and the motor 3004 is energized and rotates cam 3011 which retracts connecting rod 3006 against spring 3005. During retraction the cam follower travels along the sloping portion of the cam profile shown in FIG. 42. The reference A in FIG. 42 denotes the position of the cam follower part way through this travel. As the connecting rod retracts, the piston 3007 initially remains stationary, until the lefthand ends of the slots 3025 in connecting rod 3006 are contacted by the pins 3024 in piston 3007. The piston then travels with the connecting rod 3006 and draws degassed fluid from reservoir 3016 into a metering chamber 3031 defined in the cylinder 3026 between the valve 3019 and the lefthand end of the piston 3007. As the cam follower reaches the maximum stroke position, trip 3013 operates microswitch 3012 to switch off the motor 3004. The cam follower is now on a substantially zero lift or parallel part of the cam, and is thereby retained in a “latched” position (denoted by B in FIG. 42), and the needle-less injector is loaded and ready for use.

Referring also to FIG. 37, to cause an injection the trigger switch 3015 is depressed, and the nozzle 3020 containing orifice 3021 is placed on the subject to be injected, and pressure is applied by pushing on handle 3003 in the direction of arrow Y. The rear section 3002 is thus displaced relative to the front section 3001, and the pressure applied to the subject by nozzle 3021 is proportional to the compression of spring 3023. At a predetermined amount of displacement, a screw 3014 secured to the rear section 3002 contacts and moves trip 3013 away from the microswitch 3012. This causes the battery 3022 to be connected to the motor 3004, which then rotates the cam 3011. After a few degrees of rotation, the cam follower 3010 is suddenly released by the cam profile (reference C in FIG. 42), and the connecting rod 3006, with its mass 3029, is rapidly accelerated by the spring 3005. After traveling a distance “X” (see FIG. 37), the face 3028 on connecting rod 3006 hits the end of piston 3007 with considerable impact. The force of this impact is almost instantaneously transmitted through the degassed fluid in the metering chamber 3031, causing the degassed fluid to travel rapidly past the valve 3019 and through the orifice 3021, which is in contact with the subject. This initial impact of the degassed fluid easily pierces the epidermis of the subject, and the remainder of the piston travel completes injecting the dose of degassed fluid at relatively low pressure.

During the complete injection stroke of the connecting rod 3006, which is accomplished extremely rapidly, the cam 3011 continues rotating and picks up the cam follower 3010, thereby retracting the connecting rod 3006 until the trip 3013 contacts microswitch 3012 to turn off motor 3004. Thus the metering chamber 3031 is loaded and ready for the next injection.

The screw 3014 may be adjusted to alter the amount of displacement of section 3002 relative to section 3001 (and therefore the compression of spring 3023) before the microswitch 3012 is operated. Thus a very simple adjustment directly controls the pressure of the discharge orifice 3021 on the subject. It is necessary for the rear section 3002 to be freely movable with respect to section 3001, so that the pressure on the subject is not altered by the effects of friction.

One rotation of the cam retracts, latches and releases the spring loaded piston, and the use of the cam permits very simple, accurate and reliable operating characteristics, and a high rate of injections may be achieved with no fatigue of the operator. Furthermore, the injector operation is easy to understand and maintain by unskilled persons.

### Example 6

### Needle-Less Injector with Drive Control Mechanism

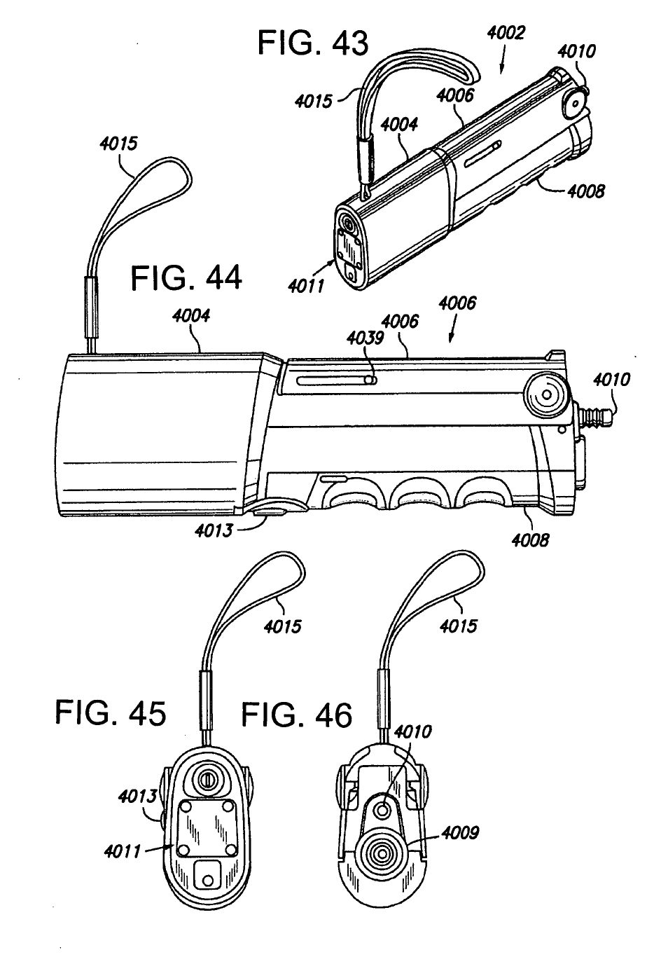

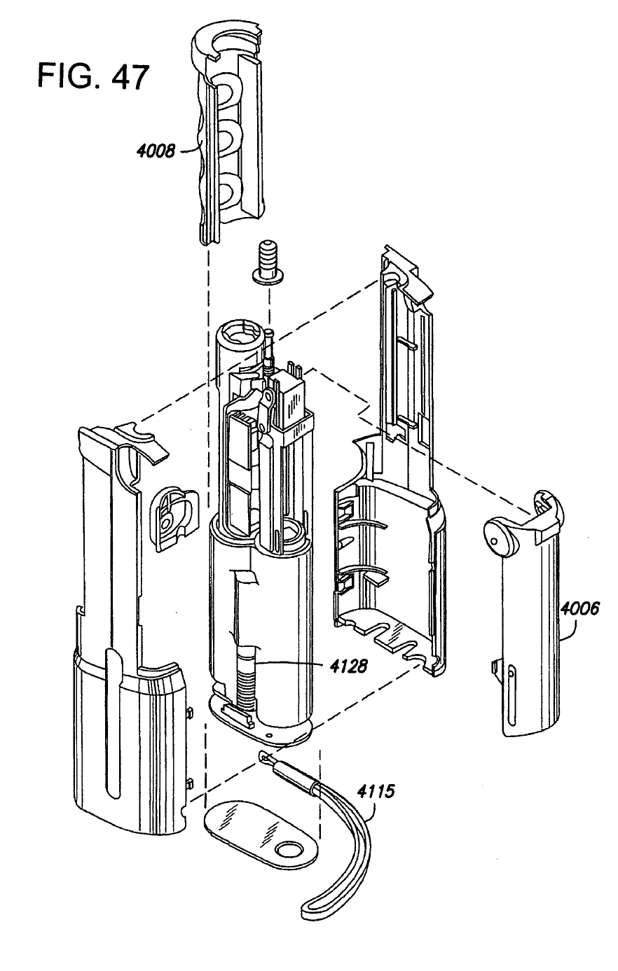

A needle-less injector is indicated generally with the numeral 4002 in FIG. 43. Referring first to FIGS. 43-47, it can be seen that needle-less injector includes a convenient molded plastic case, made up of a base portion 4004, a pivotable cartridge access door 4006, a slidable dose adjustment door 4008, a syringe collar 4009, a skin sensor 4010, an indicator panel 4011, an initiator switch 4013, and a carrying strap 4015. FIG. 47 depicts how these parts fit together to form an integral unit.

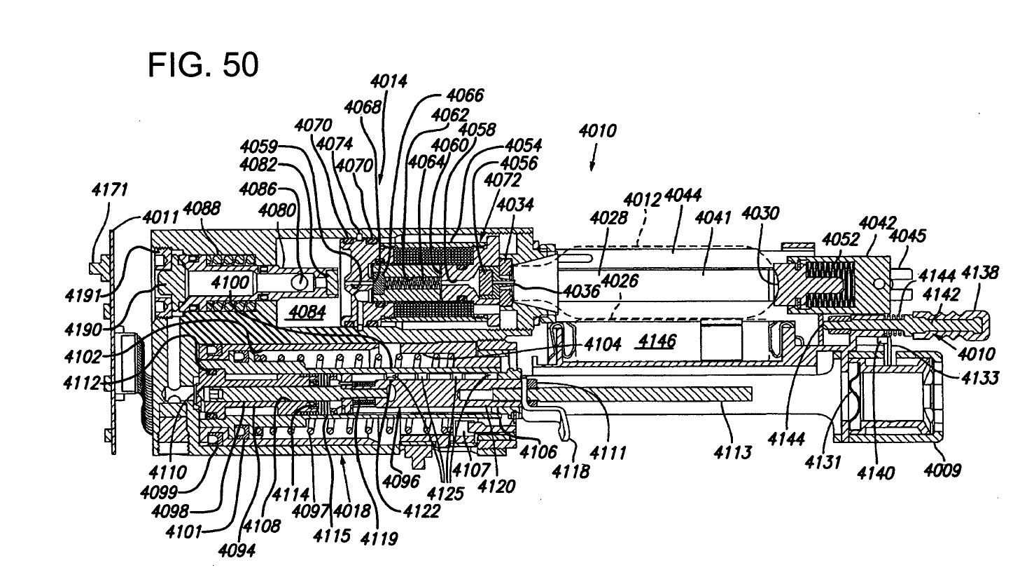

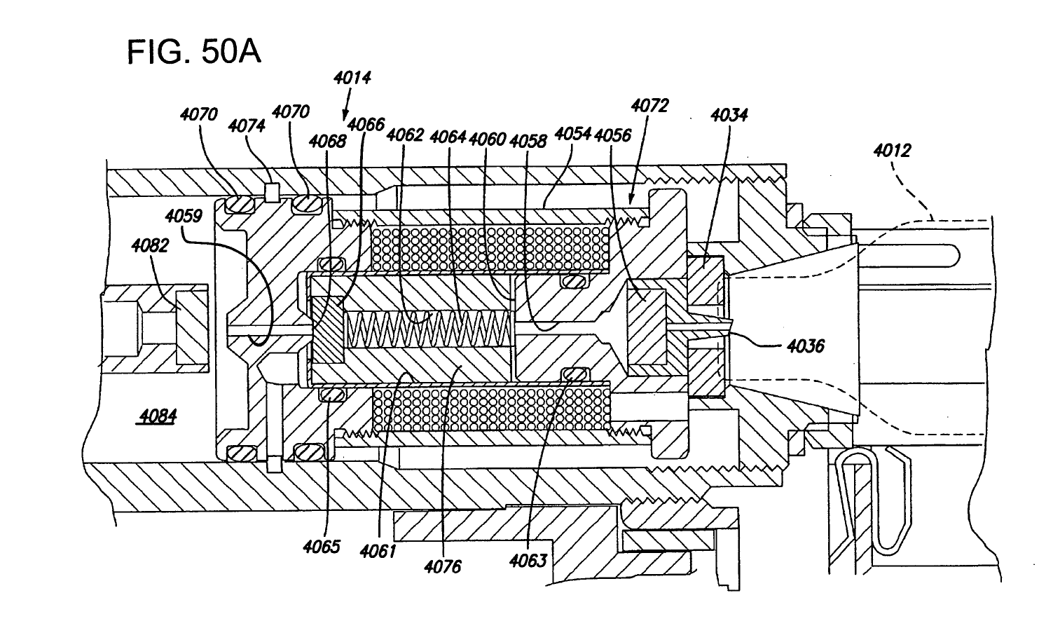

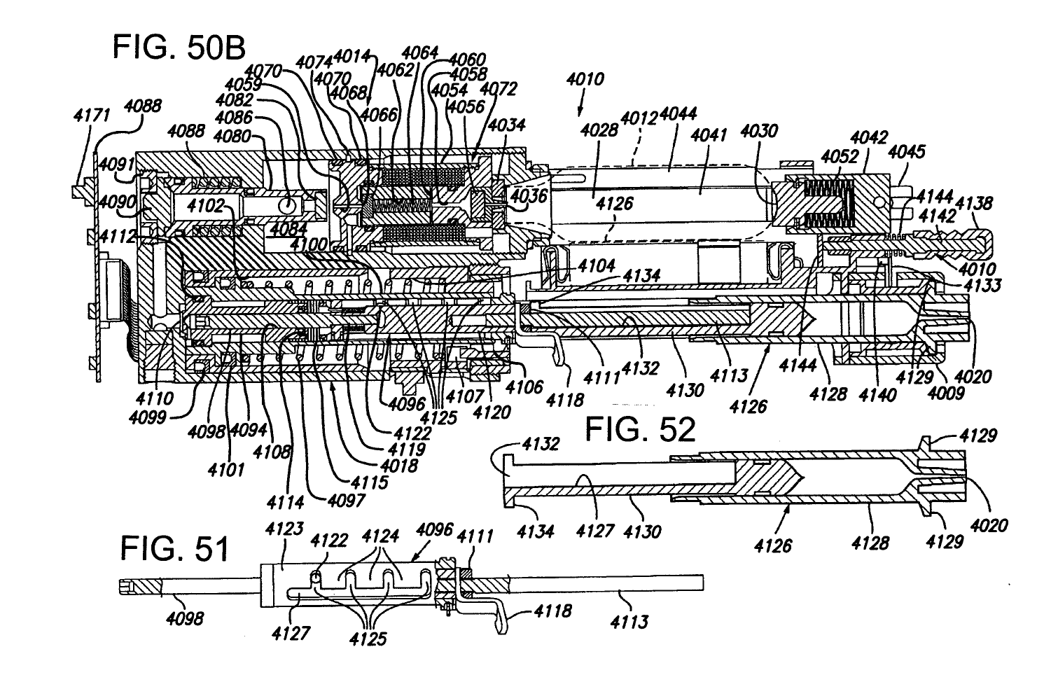

Referring now to FIG. 50, needle-less injector 4002 can be seen to include several basic components. First, a replaceable CO₂ cartridge 4012 is disposed at one side of the needle-less injector, toward the front. A cartridge pressure control system is shown behind cartridge 4012 at 4014. As shown best in FIG. 50B, disposed on the other side of the needle-less injector, at the front thereof, is a syringe 4126 which is adapted to hold and then inject a predetermined amount of degassed fluid. Positioned rearwardly of the syringe is a syringe control system 4018 which controls activation of the syringe. The syringe control system 4018 is in turn controlled from pressure which is provided by the cartridge pressure control system 4014. Indicator panel 4011 is disposed at the rear end of the needle-less injector, and it includes a power button 4171 to activate the needle-less injector and a series of indicator lamps to keep the operator advised of the condition of the needle-less injector. Skin sensor 4010 is disposed at the front end of the needle-less injector, and is used to prevent initiation of the injection process unless the skin sensor is depressed an appropriate amount as the needle-less injector is pressed against the skin of the patient. Finally, a pair of 1.5 volt AAA batteries 4026 are mounted in a battery casing 4146 disposed between CO₂ cartridge 4012 and syringe 4126 to provide power for the needle-less injector logic circuit, warning lights, etc.

The CO₂ cartridge 4012 is typically a 33 gram steel cartridge of conventional design, holding 8 grams of CO₂. This is usually enough for approximately 6-8 injections, although if the needle-less injector is used infrequently, passive gas leaks may result in fewer injections per cartridge. CO₂ cartridge 4012 is positioned within a cartridge receptacle 4028 between a forward seat 4030 which is curved to complement the curvature of the forward, rounded portion of the cartridge, and a rear area having a resilient cartridge sealing gasket 4034. This gasket is sized and positioned such that a piercing pin 4036 is adapted to extend through an annulus at the axial center of the gasket in order to pierce the rear end of the cartridge 4012 to release CO₂ pressure upon closure of hinged cartridge access door 4006.

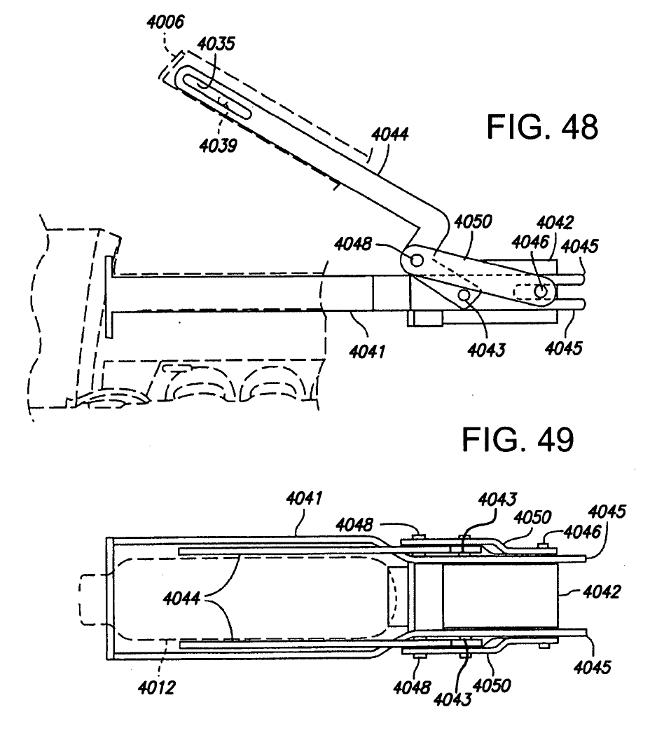

As seen in FIGS. 48 and 49, hinged cartridge access door 4006 is mounted to the ends of a pair of roughly Z-shaped cartridge door closure arms 4044 by a pair of small bolts 4039 which slide in slots 4035 as the door is opened and closed. Cartridge access door 4006 is mounted to a so-called pierce block frame 4041 and a pierce block 4042 by closure arms 4044 which straddle the pierce block and are pivotally connected to the pierce block frame at pivot points 4043. Pivot points 4043 actually are in the form of rivets, and to ensure that the pierce block travels parallel to the pierce block frame, a slot (not shown) extends along each side of the pierce block, and the inner portion of the rivet thereby guides the travel of the pierce block. Closure arms 4044 are also pivotally mounted to a pair of pivotal legs 4050 disposed to each side of pierce block frame 4041 at pivot points 4048. The opposite ends of legs 4050 pivotally connect to a pierce block pin 4046 which extends through and is mounted to pierce block 4042. Pivotable legs 4050 each include a bend at their mid-portions as shown in FIG. 49 to accommodate the length of closure arms 4044. Pierce block pin 4046 is mounted to reciprocate in a pair of forked ends 4045 in pierce block frame 4041 as cartridge access door 4006 is opened and closed and pierce block 4042 is shifted forwardly and rearwardly. Thus, when cartridge access door 4006 is closed, legs 4050 convey the motion of closure arms 4044 to pierce block pin 4046 and to pierce block 4042 which shifts within pierce block frame 4041. This causes forward seat 4030 to exert a rearward force (to the left in FIGS. 48, 49 and 50) on CO₂ cartridge 4012. As noted above, this causes piercing pin 4036 to pierce the rear end of cartridge 4012.

As best shown in FIG. 50, a series of 17 so-called belleville spring washers 4052 are disposed in series between forward seat 4030 and pierce block 4042 to provide a predetermined piercing pressure of slightly over 100 pounds, which is maintained the entire time cartridge access door 4006 is closed.

Once cartridge 4012 has been breached, pressurized CO₂ gas passes from the cartridge through piercing pin 4036, and as best shown in FIG. 50A, to a solenoid valve 4054 through a (0.25×118″2 micron) gas filter 4056 and through a conduit 4058 extending through the axial center thereof. A space 4060 extending entirely across solenoid valve 4054 thus is filled with pressurized gas, as is an axially centered spring chamber 4062 in which a solenoid spring 4064 is disposed. Solenoid spring 4064 holds a resilient solenoid seal 4066 against a solenoid seat 4068 to prevent the flow of pressure into an axially extending rear conduit 4059. A pair of O-rings 4070 are mounted in the solenoid valve to prevent flow of pressurized gas along the interior wall 4072 of the pressure control system 4014. A circumferential ring 4074 extends entirely around solenoid valve 4054 to ensure that the solenoid valve remains stationary in the pressure control system 4014.

A generally cylindrical piston 4076 is disposed between space 4060 and solenoid seal 4066. As will be described below, piston 4076, in combination with solenoid seal 4066, acts to control the flow of gas pressure through solenoid valve 4054. A sleeve 4061 fits around the piston, and well past space 4060, and an O-ring 4063 prevents CO₂ pressure from passing forwardly along the sleeve. Pressure is, however, able to pass rearwardly along the interface between the sleeve and the piston because another O-ring 4065, disposed rearwardly of space 4060, is positioned outwardly of the sleeve.

The cartridge pressure control system 4014 also includes a poppet valve 4080 (see FIG. 50) having a resilient poppet valve seal 4082 which bumps up against rear conduit 4059 to create a shuttling phenomenon when the poppet valve shifts forwardly or to the right, as will be described later. Poppet valve 4080 includes a radially extending port 4086 which interconnects the inner portion of the poppet valve with a gas reservoir 4084. Prior to the point that the reservoir is subjected to CO₂ pressure, poppet valve 4080 will be in the position depicted in FIG. 50. A poppet valve spring 4088 holds the poppet valve in the depicted position, with a poppet valve seat 4090 disposed against the poppet valve to close the poppet valve.

Upon closure of cartridge access door 4006, and with the solenoid valve in the depicted position, pressurized CO₂ flows through piercing pin 4036 and filter 4056 (see FIG. 50A). It is directed through conduit 4058 and into space 4060 and spring chamber 4062, and along the interface between sleeve 4061 and piston 4076 to the rear of the piston. While the pressure is therefore equalized at the two ends of the piston, because the surface area is greater on the front side of the piston if the surface area of solenoid seal 66 is included, the piston will remain in the position shown in FIGS. 50 and 50A, with the solenoid seal seated firmly against solenoid seat 4068, thereby preventing pressure from entering reservoir 4084.

Once the needle-less injector is initiated to inject degassed fluid, solenoid valve 4054 is shifted slightly (approximately 0.012 inch) forward or to the right in FIGS. 50 and 50A, but not so far as to close off space 4060. This enables pressurized gas to flow through rear conduit 4059 into gas reservoir 4084.

From the gas reservoir, pressurized CO₂ flows through port 4086 in poppet valve 4080 (see FIG. 50). When the increased pressure within the poppet valve causes the upward force on the poppet valve to exceed the rearward or leftward force of poppet valve spring 4088, the poppet valve lifts off its seat 4090, permitting pressure to rush into the next section of the needle-less injector. The poppet valve is normally set to lift off of its seat at a pressure of 480 psi. When poppet valve 4080 is in this raised position, poppet valve seal 4082 bumps up against rear conduit 4059. When the poppet valve opens, the pressure in gas reservoir drops, so that the force of poppet valve spring 4088 again exceeds the force of pressurized gas, thereby causing the poppet valve to close. This in turn permits the pressure in gas reservoir 4084 to immediately increase, lifting the poppet valve again. This phenomenon, called shuttling, continues for a short period of time until the degassed fluid is fully injected. Normally the controller closes the solenoid valve 0.8 seconds after it is opened, so that the time of termination of the shuttling is determined by the controller.

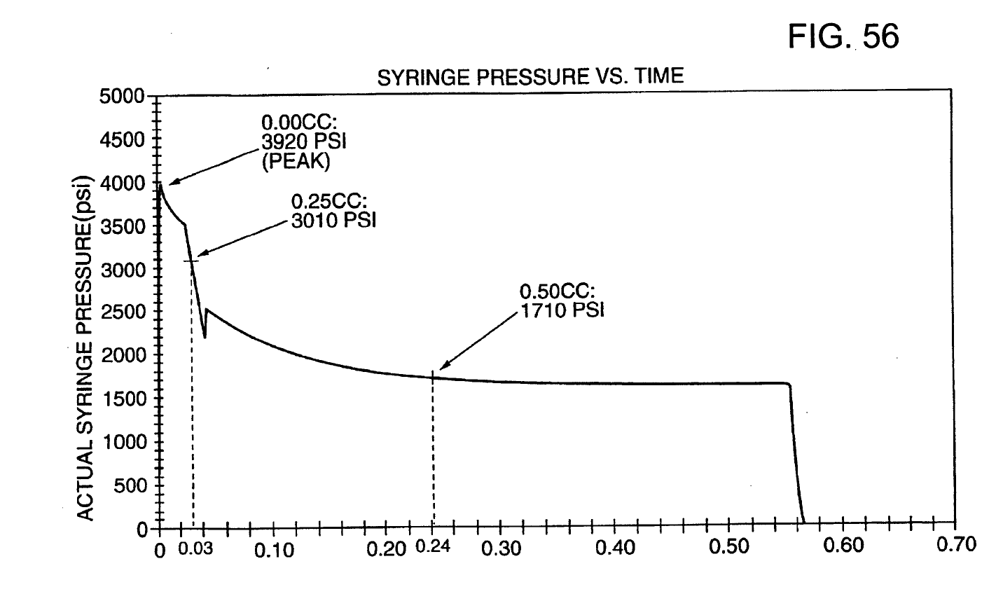

The initial rush of pressure followed by shuttling produces a pressure profile which is ideal for a needle-less injection system. As shown in FIG. 56, the initial rush of CO₂ pressure provides a syringe pressure of approximately 3920 psi to penetrate the patient's skin, followed by a sustained, substantially constant pressure of approximately 1700 psi for about 0.5 seconds during the shuttling phase. The term “substantially constant” as used herein is intended to encompass a variation of from about 2000 psi to 1600 psi as shown in FIG. 56 between the 0.1 and 0.56 second points of the injection cycle. This pressure profile has been found to be superior to some prior art pressure profiles which peak quickly but then drop off sharply. Assuming 1.0 cc is injected, it can be seen that approximately 0.25 cc is injected at the higher pressures, but much more than half of the degassed fluid is injected during the shuttling, lower pressure phase.

A threaded poppet valve pressure adjustment face 4091 may be threaded inwardly to increase or outwardly to decrease the pressure at which poppet valve 4080 opens and closes. A special tool (not shown) is used to facilitate this adjustment.

Referring to FIGS. 50 and 50A, the syringe control system 4018, which receives CO₂ pressure from poppet valve 4080, will now be described. This system includes a dose compensator cylinder 4094, a dose variation assembly 4096 having a pressure piston 4098 mounted thereto, an inner cylinder 4100, a rearward outer cylinder 4102, and a forward outer cylinder 4104. So-called U-cup seals 4099 and 4101 will prevent pressure leakage between the stages of the syringe control system. The CO₂ pressure entering the syringe control system 4018 causes the entire rearward outer cylinder 4102 to shift forwardly or to the right in FIG. 50, against the compressive action of a light helical spring 4097. Rearward outer cylinder 4102 continues to shift until its forward end contacts the rear end of forward outer cylinder 4104, which is about ⅛- 3/16 inch into its travel. At this point, inner cylinder 4100 continues to move in a forward direction for approximately another 1½ inch, for a total travel of approximately 1⅛ inches. This independent movement of the inner cylinder generally corresponds with the point that the shuttling begins in the cartridge pressure control system 4014. Thus, the independent movement of inner cylinder 4100 cooperates with the shuttling action to provide a reduced, substantially constant but lower second pressure phase to the needle-less injector. At this point, spring 4097 will have bottomed out and immediately thereafter the controller will cause the solenoid valve to shut off CO₂ pressure.

Dose compensator cylinder 4094 travels with inner cylinder 4100 and rearward outer cylinder 4102 in their above-described forward motion. Dose compensator cylinder 4094 is a generally cylindrical member having a soft rubber bumper at the rear end thereof (not shown due to its small dimensions), and a centrally disposed axially extending channel 4108 with an entry segment 4110 at the rear end thereof, as shown best in FIG. 50. This entry segment 4110 selectively interconnects channel 4108 with fluid pressure from poppet valve 4080. An O-ring 4112 is provided on dose compensator cylinder 4094 to prevent the flow of fluid pressure along the outer surface of the cylinder. A seal 4114 is provided at the forward end of the dose compensator cylinder to minimize any leakage between the inner cylinder wall defining channel 4108 and pressure piston 4098.

The purpose of the dose compensator cylinder system is to account for the fact that pressure will tend to act somewhat differently on the syringe control system 4018 when there is a greater or lesser amount of degassed fluid in the syringe. Because pressure piston 4098 will move forwardly and rearwardly within channel 4108 as the dosage is decreased and increased, respectively, thereby increasing and decreasing, respectively, the size of a chamber defined within channel 4108 behind piston 4098, this accommodation is made.

A helical spring 4115 is positioned between dose compensator cylinder 4094 and dose variation assembly 4096 as shown in FIG. 50. Spring 4115 provides a suitable amount of pressure which is passed onto syringe 4126 and the degassed fluid provided therein to make sure that no air is in the system. With such a forward biasing of the syringe, the amount of degassed fluid in the syringe can be measured. As will be described below, if the dose variation assembly 4096 is too far forward or to the right in FIG. 50, which indicates that there is an insufficient amount of degassed fluid in the syringe, then an interlock will prevent the needle-less injector from firing. This condition is sensed by a dose indicator flag 4106 disposed in a dose indicator optical interrupter space 4107. Dose indicator flag 4106 is mounted to a cylindrical dose variation compensator 4120 so that the position of the flag generally corresponds to the amount of degassed fluid in the syringe. When there is sufficient degassed fluid in the syringe, flag 4106 will block infrared light from passing across space 4107 from an illuminator (not shown) to a receptor (not shown). When there is an insufficient amount of degassed fluid in the syringe, spring 4115 will cause flag 4106 to be shifted to the right, withdrawing the flag from space 4107 and permitting infrared light to pass from the illuminator to the receptor, which will send a signal to the controller, thereby lighting a warning lamp and preventing the needle-less injector from entering its initiation phase. It is possible that micro switches, magnetic switches, or other conventional position sensors (not shown) may be utilized in place of the optical interrupter described above.

Dose variation assembly 4096 permits the dosage to easily be adjusted in ¼ cc increments (see FIG. 51). This is done through the use of a thumb-nail manipulator 4118 which extends radially outwardly from the unit and which is mounted by a lock nut 4111 to an axially extending rod 4113 which is threaded into dose variation compensator 4120. The dose variation compensator has a generally semi-spherical protrusion 4122 mounted on it, and it is surrounded by a cylindrical jacket 4123 shown best in FIG. 51. This jacket 4123 has four circumferentially extending slots 4125 interconnected by a single axially extending slot 4127, the four slots being adapted to selectively receive semi-circular protrusion 4122. Partitions 4124 are disposed between and define the four slots. FIG. 51 shows that the partitions are relatively narrow in their circumferential dimensions, so that with only a 40°-45° twist of compensator 4120 with thumbnail manipulator 4118, semi-spherical protrusion 4122 can clear the adjacent partition, and under the pressure from springs 4115, will be biased forwardly through axially extending slot 4127 into the next adjacent slot 4125, thereby adjusting the dosage by 3¼ cc. If the thumbnail manipulator 4118 has not been released, then the protrusion can selectively be guided over to another one of the four slots, depending upon the desired dosage. Once positioned, releasing the thumbnail manipulator permits a series of rotational biasing springs 4119 to cause dose variation compensator 4120 to rotate, which in turn moves the protrusion into one of the four slots 4125.

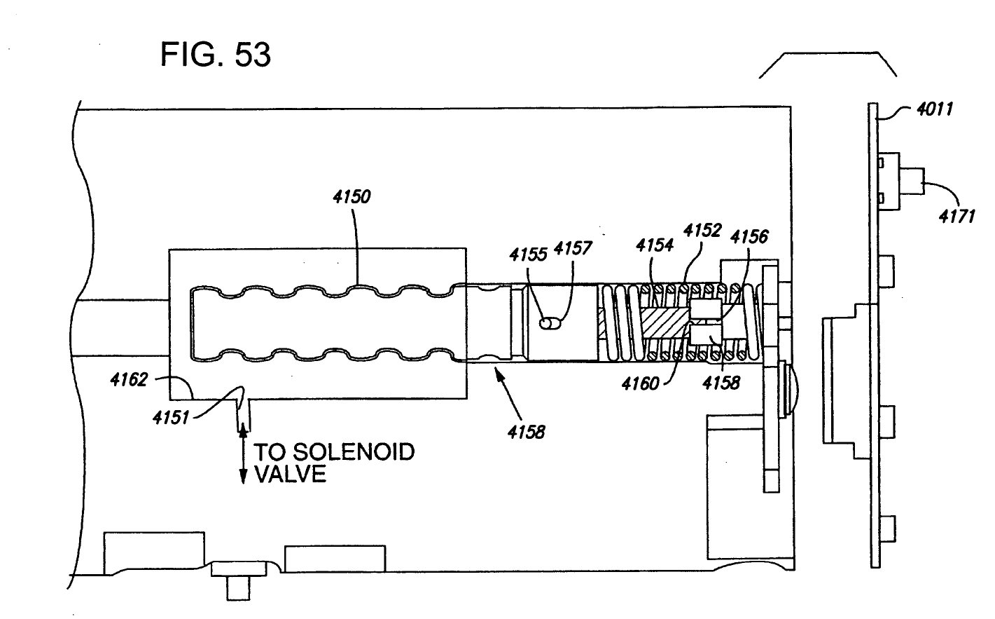

The syringe 4126 is shown best in FIG. 52. It includes an ampoule 4128 and a plunger 4130. The end of the plunger includes a radially extending notch 4132 which is interconnected with an axial slot 4127 which is sized to fit onto rod 4113 extending from dose variation compensator 4120. A flared end 4134 on the plunger is designed to abut the forward end of dose variation compensator 4120. Thus, the axial drive force imparted to the compensator by rearward outer cylinder 4102 and inner cylinder 4100 will cause the plunger to drive forwardly, forcing degassed fluid out of the syringe. The syringe also includes a pair of opposed flanges 4129 disposed adjacent the forward end thereof. The syringe ampoule 4128 includes a small injection aperture 4020 at the forward end. Aperture 4020 is typically 0.0045 inch in diameter, although it may be as large as 0.014 inch, depending upon the subcutaneous injection depth which is desired.

Syringe 4126 fits into the needle-less injector by merely inserting the syringe through collar 4009 in the front end of the needle-less injector, and pushing it in. When it is most of the way in, pressure from spring 4115 will be felt. When it bottoms out against a wave spring 4131, the syringe is rotated approximately 90° so that flanges 4129 are engaged within the syringe collar 4009 as shown in FIG. 50B. As the syringe is rotated that 90°, it engages a pin 4133 which rotates with it. Once this pin 4133 is rotated, it depresses a syringe lock micro switch 4140, which sends a signal to the controller that the syringe has been properly installed. If this syringe lock micro switch is not depressed, the controller will light a warning lamp and prevent the needle-less injector from entering its initiation phase.

A pressure switch 4148 is disposed midway between and to the side of the portions of the needle-less injector which house the cartridge pressure control system 4014 and the syringe control system 4018, as shown best in FIG. 47. Referring now to FIG. 53, pressure switch 4148 includes a bellows 4150, a spring 4152 and a central rod 4154 which terminates in a flag 4156. Flag 4156 is disposed within a stationary optical interrupter 4158 which transmits infrared light across a space 4160 much like the previously-described dose measurement optical interrupter. When the flag is disposed within the space, the light is interrupted and a collector (not shown), which otherwise receives light from an emitter (not shown), sends a signal to a controller.

Bellows 4150 is subjected to CO₂ cartridge pressure because a port 4151 interconnects an otherwise-sealed chamber 4162 surrounding the bellows with the CO₂ pressure present within solenoid valve 4054. The variations in pressure cause the bellows to expand and contract, causing rod 4154 and flag 4156 to move slightly forwardly and rearwardly in relation to optical interrupter 4158. A pin 4155 travels within a short slot 4157 such that some contraction or expansion of the bellows is permitted without causing any displacement of flag 4156. If the pressure is relatively high, the flag blocks the transmission of infrared light across space 4160, but if the pressure is not as high as it should be, spring 4152 causes bellows 4150 to extend slightly into chamber 4162, thereby causing rod 4154 to withdraw flag 4156 from optical interrupter 4158, permitting infrared light to be conveyed to a collector. This sends a signal to the controller, which lights an appropriate warning lamp and terminates the initiation cycle.

It is possible that a pressure switch other than the above-described bellows/optical interrupter could be used. For example, it may be possible to use a helical or a spiral bourdon tube could be used in place of the bellows, and another type of switch other than the described optical interrupter.

To ensure that the needle-less injector is pressed up against the skin of the patient prior to activation of the needle-less injector, skin sensor 4010 is provided. The skin sensor includes an extension rod 4142 which is forwardly biased under the pressure of a skin sensor spring 4144 to the extended position shown in FIG. 50. A soft plastic jacket 4138 fits over the extension rod in the depicted embodiment. As the needle-less injector is sufficiently pressed against the skin of the patient, the extension rod is depressed against the pressure of the skin sensor spring, and a spring sensor micro switch 4144 is contacted, sending an electronic signal to the controller to prevent termination of the initiation cycle. If the skin sensor is not sufficiently depressed, the controller lights a warning lamp and the initiation cycle is terminated. Skin sensor 4010 thereby functions to prevent inadvertent or other discharge of the needle-less injector when the needle-less injector is not properly positioned against the skin, which may happen if the patient is reluctant or, again, physical dexterity problems make it difficult for the patient to properly position the needle-less injector.

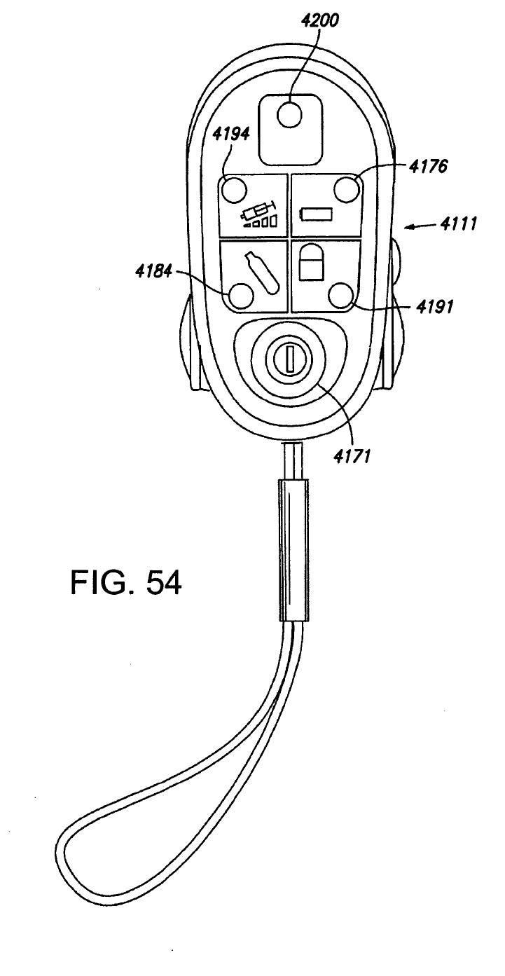

Indicator panel 4011, shown best in FIG. 54, includes the following red warning lamps: CO₂ pressure warning lamp 4184; dose volume warning lamp 4194; syringe lock warning lamp 4191; and battery warning lamp 4176. A green “ready” lamp 4200 is also included, as is a power button 4171.

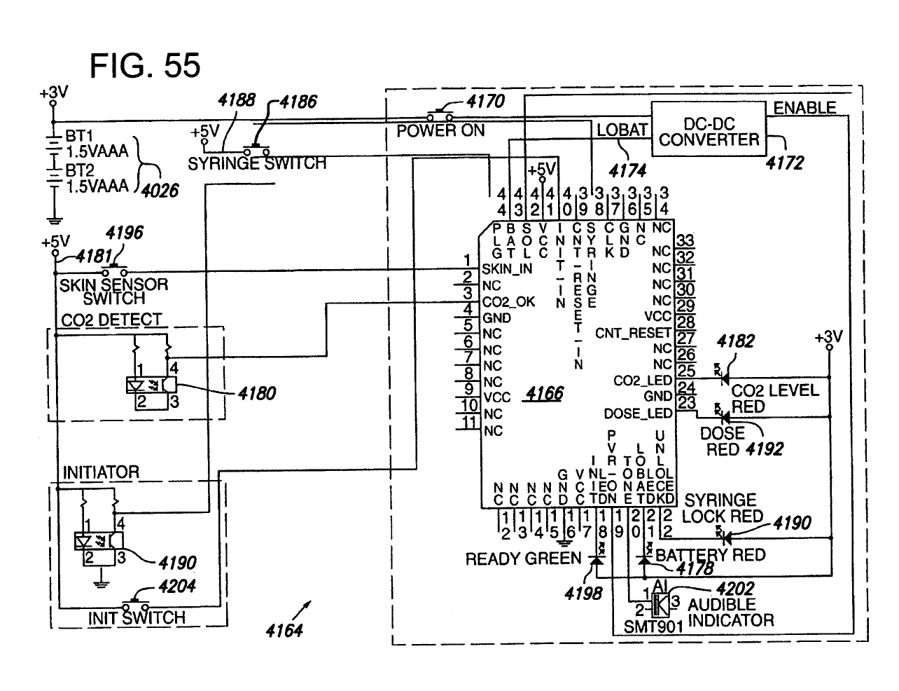

Reference will be made to the control circuit schematic, FIG. 55, as well as to the indicator panel 4011 provided at the rear of the needle-less injector, and depicted in FIG. 54. The logic circuit, indicated generally with the numeral 4164, selectively provides power to light the lamps of the indicator panel. Central to the circuit is controller 4166 which in the preferred embodiment is an Atmel programmable logic device, designated as model ATF 1500L. This is a low power unit which can effectively control the operation of the needle-less injector while using a minimal amount of power so that the batteries do not have to be replaced very often.

As mentioned earlier, the needle-less injector includes a number of interlocks which prevent the unit from operating, and warn the operator in the event any one of a number of conditions is not satisfied. The logic circuit provides this capability, but before describing those features, reference will first be made to the general layout of the circuit.

The batteries, shown at 4026, are mounted in series to provide 3 volts of DC power to the circuit. A power switch 4070, which corresponds with power button 4171 (see FIG. 54), controls the flow of power to a DC-DC converter 4172, which converts the 3 volt charge to a 5 volt charge as needed elsewhere in the circuit. In the event there is low battery power, a signal is sent to the controller via line 4174, and a red “battery” light 4176 is activated in indicator panel 4011 depicted in FIG. 54. This light is energized by an LED 4178 which is connected to an active low pin in controller 4166 which sends the 3 volt charge to ground upon a low battery signal from line 4174, thereby energizing the LED and warning the operator that the batteries need to be replaced. This event prevents the initiation of the needle-less injector so that even if the operator ignores the light, the needle-less injector cannot be initiated. In the event there is sufficient battery power, voltage is provided to the controller to activate the needle-less injector.

Another one of the interlocks provides protection for insufficient CO₂ pressure. As described above, pressure switch 4148 determines whether sufficient CO₂ pressure is sufficient. If it is, flag 4156 will block light from passing through optical interrupter 4158, and a transistor in a CO₂ detect subcircuit 4180 will remain open. In this condition, the controller will sense the 5 volt charge coming in from line 4181. If CO₂ pressure is insufficient, the flag will withdraw, permitting light to pass through the optical interrupter, which will then close the CO₂ detect subcircuit 4180, thus grounding the 5 volt charge, which will be sensed by the controller. Simultaneously, an active low pin to which a CO₂ cartridge LED 4182 is connected will ground that LED circuit, energizing that LED and activating a red CO₂ cartridge light 4184 in indicator panel 4011, as shown in FIG. 54. This event also causes the controller to prevent initiation of the needle-less injector even if the user ignores the indicator panel warning light 4184.

Yet another interlock is provided to ensure that there is sufficient degassed fluid in ampoule 4128. A dose detect subcircuit 4190, very similar to CO₂ detect subcircuit 4180, is provided. Dose detect subcircuit 4190 is provided with a 5 volt charge from line 4181, and if a sufficient dosage level is sensed by the dose indicator optical interrupter, a transistor in the dose detector subcircuit will remain open and the controller will sense 5 volts. If the dose is insufficient, the transistor will close and the controller will sense the absence of the 5 volt charge. In that event, a red volume warning lamp 4194 is lit in indicator panel 4011 by a dose detector LED 4192. This light is connected to an active low pin in the controller which sends the 3 volt charge to ground, thereby activating the LED. Unless the dose is sufficient, this dose interlock will warn the user at the indicator panel and will prevent the needle-less injector from initiating.

Yet another interlock is provided with a syringe switch 4186 which ensures that the syringe is properly locked into positioned in the needle-less injector before the unit is initiated. As noted previously, this condition is sensed by syringe lock micro switch 4140. Syringe switch 4186 receives a 5 volt charge from line 4188. If the syringe is properly locked in place, the syringe switch will be closed. In this condition, the controller senses the 5 volt charge, and the needle-less injector is ready for initiation. If the syringe is not properly locked in place, a syringe lock LED 4193 will activate a red syringe lock warning lamp 4191 and the controller will prevent the needle-less injector from entering its initiation cycle.

If all of the conditions have been met (other than the next-to-be-described skin sensor), the controller operates to flash a green “ready” light 4200 in indicator panel 4011.

The skin sensor interlock will now be described. A skin sensor switch 4196 is provided off 5 volt line 4181. In order to initiate the needle-less injector, skin sensor 4010 must be depressed, thereby closing skin sensor switch 4196 and sending a 5 volt charge to the controller. Unless this charge is sensed by the controller, the needle-less injector will be prevented from entering its initiation cycle. When the charge is received, showing that everything is ready for initiation, skin sensor LED 4198 will provide a steady activation of the green “ready” light 4200 in indicator panel 4011, and an audible indicator 4202 will emit a beep.

An initiator switch 4204 is also provided off 5 volt line 4181, which is closed by depressing indicator panel initiator button 4171. If all of the foregoing conditions have been satisfied, closing of the initiator switch will send a 5 volt charge to the controller, which in turn sends power to solenoid valve 4054 to cause CO₂ pressure to inject degassed fluid into the patient. If any of the foregoing conditions have not been satisfied, the appropriate warning lights will be lit, and the controller will prevent the needle-less injector from entering its initiation cycle.

### Example 7

### Needle-Less Injector Including a Lyophilized Product

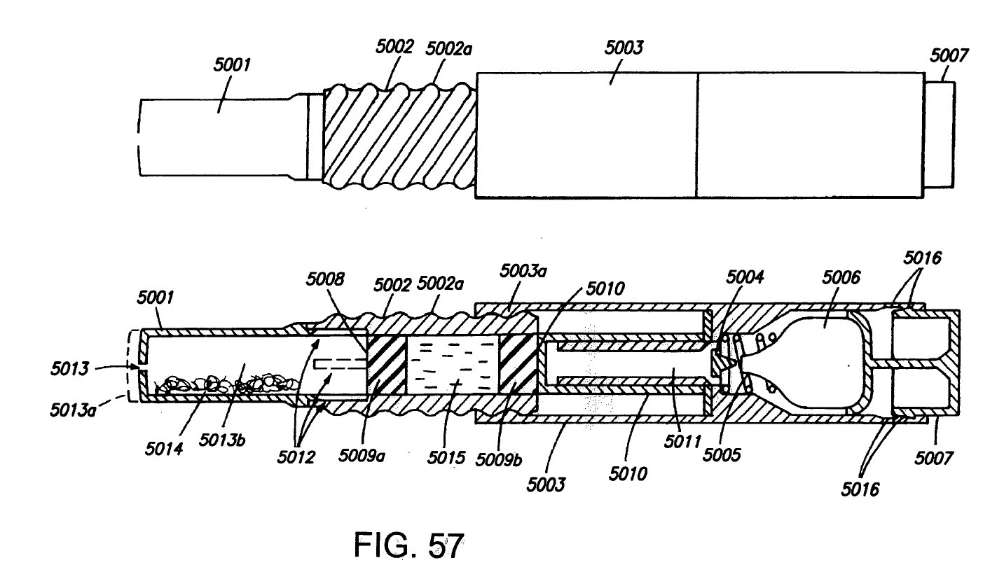

Referring to FIG. 57, an embodiment of a needle-less injector includes three sections herein referred to as lower 5001, middle 5002, and upper 5003. With the exception of the moisture resistant, e.g., metal foil, seals, spring 5005 and compressed gas reservoir 5006, all the other parts can be manufactured by plastic injection molding.

The lower section includes cylindrical housing 5001, further defined by orifice 5013, cap 5013a, and one or a plurality, e.g., three or four, evenly spaced grooves 5012 on the inside of 5001 at the end facing piston 5009a. The space 5013b within 5001 is reserved for lyophilized product 5014.

The middle section is characterized by cylindrical housing 5002 having an exterior thread 5002a and is further defined by a fluid reservoir 5015 containing a degassed fluid 5015a. Fluid reservoir 5015 is bounded by two pistons, e.g., rigid pistons having elastomeric seals or elastomeric pistons 5009a,b having metal foil seals on the outside aspect of housing 5002.

Housing 5002 may be manufactured separately from housing 5001 such that it can be further characterized by a vapor deposited metal film on its outer surface (vapor barrier metalization is desirable if the material does not have a suitable vapor transmission characteristics). Housing 5001 and 5002 must be securely mated at the time of assembly. This 2-part assembly allows for visual inspection of the mixing of degassed fluid 5015a with lyophilized product 5014 while at the same time providing a vapor transmission barrier around the contained degassed fluid 5015. The metallized vapor barrier consisting of the metal foil seals on the outer ends of plungers 5009a,b and the coating on the outside of housing 5002 will aid in ensuring a long shelf-life for the lyophilized product. In addition to glass, metal foils and coatings offer the best protection against water vapor transmission. Since the needle-less injector assembly may be packaged in a foil pouch, any water vapor escaping from the fluid reservoir will accumulate within the air inside the foil pouch. This accumulated water vapor may have an adverse effect on the stability of the lyophilized product. This can be prevented or greatly reduced by the all encompassing metal barrier surrounding fluid reservoir 5015.

The upper section includes cylindrical housing 5003 having floating plunger 5010, a space 5011, fixed actuator 5004, spring 5005, compressed gas reservoir 5006, release button 5007 and detents 5016. Housing 5003 is further characterized by a thread 5003a on the inside of the housing which mates with that 5002a on the outside of middle section 5002.

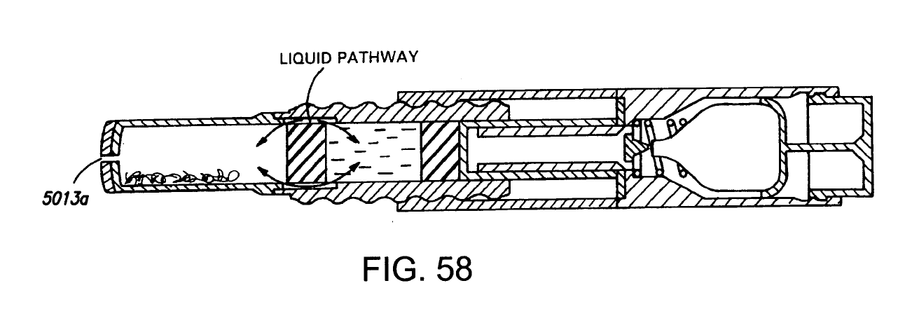

Referring to FIGS. 58, 59 and 60, use of the device is described. The device is removed from its foil pouch. The foil seal is removed from housing 5001 and assembled with housing 5002 (in some embodiments the foil seal is pierced automatically when the chambers are engaged). Holding the needle-less injector assembly in the vertical position with orifice 5013 pointing up, grasp the lower section 5002 (end facing up) with one hand and with the other rotate housing 5003 around housing 5002. This action results in floating plunger 5010 pushing against plunger 5009b thereby pushing degassed fluid column 5015a and plunger 5009a into the space defined by grooves 5012. Pistons 9a and 9b are under radial compression. Since plunger 5009a is under compression when assembled, it expands when it enters the space surrounding grooves 5012 thereby providing resistance to further movement. This is depicted in FIG. 58. The hydraulic coupling between the two pistons 5009a and 5009b is removed once the piston 5009a is positioned with the grooves around it allowing the degassed fluid to transfer to chamber 5001. As housing 5003 is further rotated, degassed fluid 5015a flows by piston 5009a through grooves 5012 and into space 5013b containing lyophilized product 5014 until all the degassed fluid is pushed into housing 5001 at which time housing 5003 reaches the end of its travel (e.g., approximately ¾ turn, the amount of rotation can vary, e.g., on the thread pitch selected). This is depicted in FIG. 49. The air displaced by degassed fluid 5015a escapes through the hydrophobic vent in cap 5013a. In this position piston 5009a and 5009b have made contact and jointly form a seal over the fluid transfer slots. The needle-less injector assembly is rocked in a back and forth motion until the lyophilized product is totally dissolved and thoroughly mixed with degassed fluid 5015a.

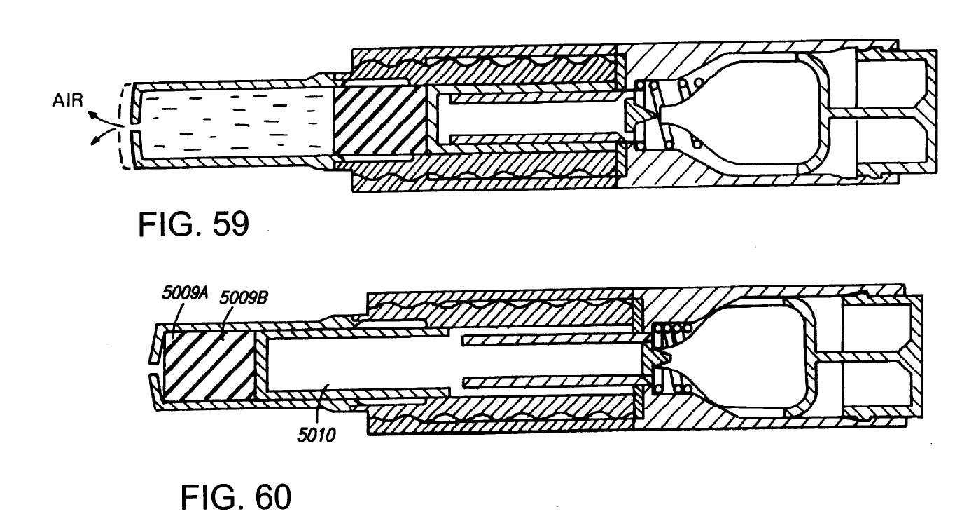

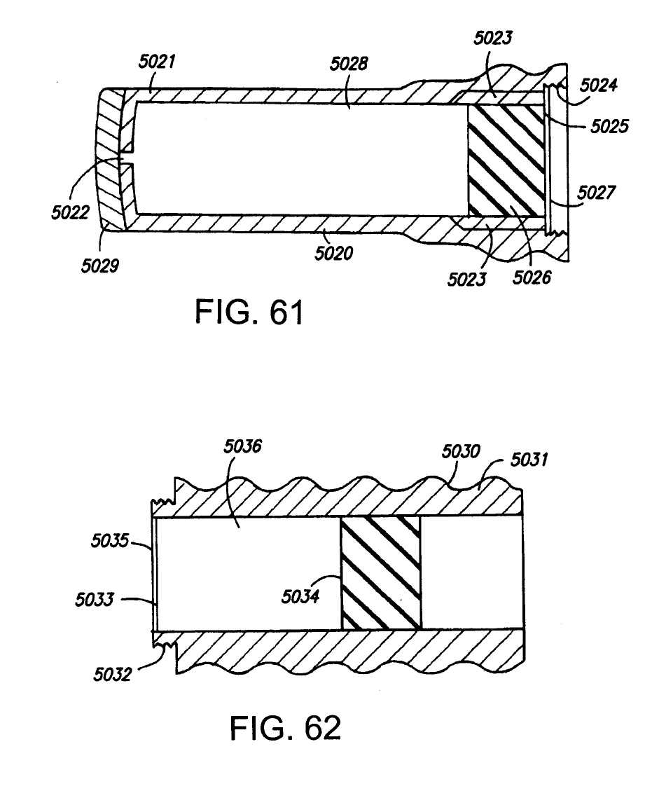

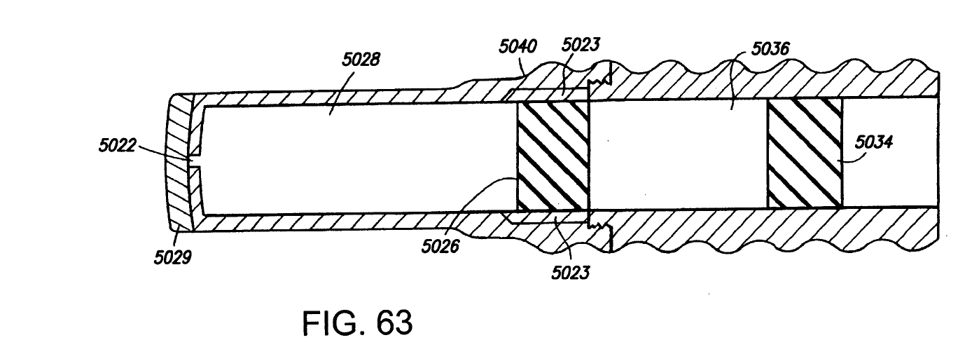

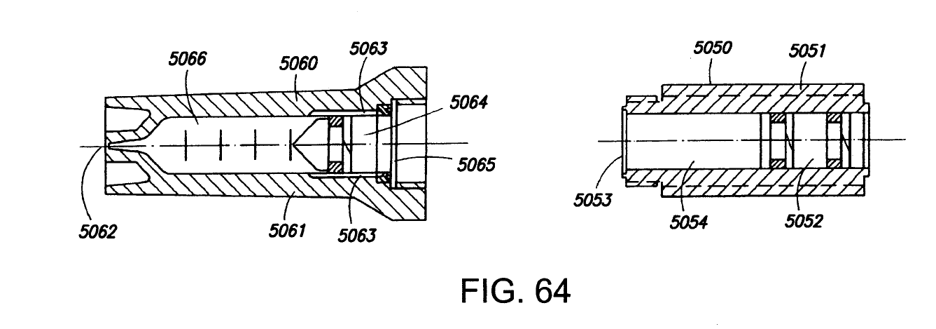

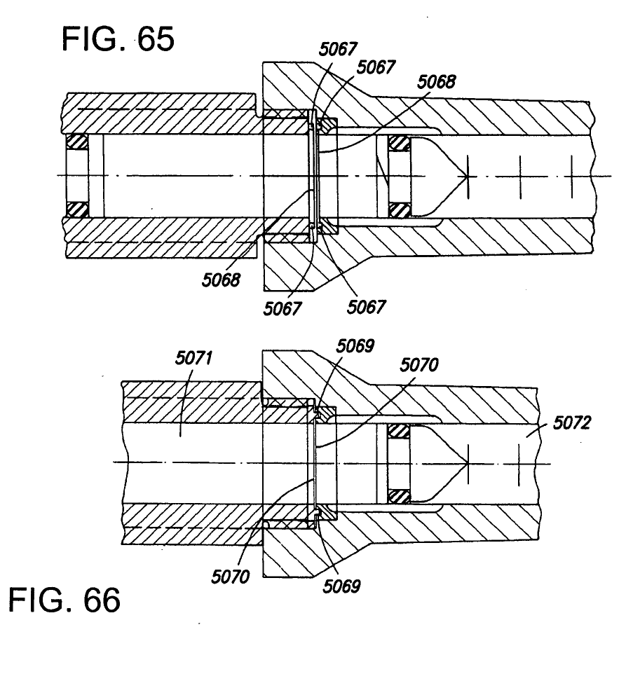

To inject the mixture of the lyophilized product and degassed fluid into the body, cap 5013a is removed and while holding the needle-less injector assembly in the vertical position, orifice 5013 is pressed against the skin. The thumb is then used to press injection button 5007. This action locks the button in position at detents 5016, and actuator 5004 seats against the chambered end of space 5011. When gas reservoir 5006 hits the pointed end of the actuator 5004, a seal is ruptured in reservoir 5006 thereby releasing the compressed gas contained therein. The gas escapes through actuator 5004 and into space 5011 where it impinges upon the bottom of floating plunger 5010. Plunger 5010 pushes against mated pistons 5009a,b (see FIG. 59) thereby expelling the mixture through orifice 5013 and into the skin. The entire injection process is complete less than 2 seconds. The final position of the pistons is depicted in FIG. 60. At this point, the injection is complete and the needle-less injector is ready for disposal.

In another embodiment, the gas pressure can be generated by a chemical reaction similar to that found in automobile air bags. This chemical reaction is extremely fast and efficient and creates a source of high-pressure nitrogen gas. Furthermore, the chambers which hold the two substances can be provided by separate modules. The lower 5001 and middle 5002 sections of FIG. 57 can be replaced with the modular components.

While the description above refers to particular embodiments of the present invention, it should be readily apparent to people of ordinary skill in the art that a number of modifications may be made without departing from the spirit thereof. Specifically, there is a wide array of needle-less injectors and other needle-less injection devices that may be suitable for use in accordance with the present invention. Most, if not all needle-less injectors and other needle-less injection devices may be filled with a degassed fluid, and used accordingly.

The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

1. A needle-less injector to administer an injection of a degassed fluid, said needle-less injector containing a degassed fluid.

2. The needle-less injector of claim 1, further comprising an ampoule to contain said degassed fluid.

3. The needle-less injector of claim 2, wherein said ampoule is configured to be filled with said degassed fluid and stored separate from remaining components of said needle-less injector prior to use of said needle-less injector to administer a needle-less injection.

4. The needle-less injector of claim 1, wherein upon actuation of said needle-less injector said degassed fluid is entirely evacuated from said needle-less injector.

5. The needle-less injector of claim 1, wherein upon actuation of said needle-less injector said degassed fluid is partially evacuated from said needle-less injector.

6. The needle-less injector of claim 1, wherein said degassed fluid is selected from the group consisting of liquids, solutions, suspensions, mixtures, diluents, reagents, solvents, emulsions, pharmaceutical vehicles, pharmaceutical excipients, vaccines, injectable medications, drugs, pharmaceutical agents, nucleotide based medications, saline solution, non-medicinal fluids administered as a placebo in a clinical study, two-component injectates, and combinations thereof.

7. The needle-less injector of claim 1, wherein said needle-less injector is powered by a power source selected from the group consisting of a spring, pressurized gas, electricity, and combinations thereof.

8. The needle-less injector of claim 1, said needle-less injector containing a lyophilized product to mix with said degassed fluid.

9. A method of administering a needle-less injection of a degassed fluid to a recipient, comprising:
providing a needle-less injector containing a degassed fluid; and
administering a needle-less injection of said degassed fluid with said needle-less injector to said recipient.

10. The method of claim 9, wherein providing a needle-less injector containing a degassed fluid further comprises:
mating an ampoule containing said degassed fluid to remaining components of said needle-less injector.

11. The method of claim 9, wherein administering said needle-less injection of said degassed fluid to said recipient further comprises:
entirely evacuating said degassed fluid from said needle-less injector into said recipient.

12. The method of claim 9, wherein administering said needle-less injection of said degassed fluid to said recipient further comprises:
partially evacuating said degassed fluid from said needle-less injector into said recipient.

13. The method of claim 9, wherein said degassed fluid is selected from the group consisting of liquids, solutions, suspensions, mixtures, diluents, reagents, solvents, emulsions, pharmaceutical vehicles, pharmaceutical excipients, vaccines, injectable medications, drugs, pharmaceutical agents, nucleotide based medications, saline solution, non-medicinal fluids administered as a placebo in a clinical study, two-component injectates, and combinations thereof.

14. The method of claim 9, wherein said needle-less injector is powered by a power source selected from the group consisting of a spring, pressurized gas, electricity, and combinations thereof.

15. The method of claim 9, wherein administering said needle-less injection of said degassed fluid with said needle-less injector to said recipient further comprises:
mixing said degassed fluid with a lyophilized product contained in said needle-less injector to create a mixture; and
administering said mixture to said recipient.

16. A method of providing a needle-less injector filled with an injectate that is substantially free of gas pockets, comprising:
providing an injectate that is a degassed fluid; and
filling said needle-less injector with said injectate.

17. The method of claim 16, wherein filling said needle-less injector with said injectate further comprises:
providing an ampoule;
filling said ampoule with said injectate; and
mating said ampoule to remaining components of said needle-less injector.

18. The method of claim 16, wherein said degassed fluid is selected from the group consisting of liquids, solutions, suspensions, mixtures, diluents, reagents, solvents, emulsions, pharmaceutical vehicles, pharmaceutical excipients, vaccines, injectable medications, drugs, pharmaceutical agents, nucleotide based medications, saline solution, non-medicinal fluids administered as a placebo in a clinical study, two-component injectates, and combinations thereof.

19. The method of claim 16, wherein said needle-less injector is powered by a power source selected from the group consisting of a spring, pressurized gas, electricity, and combinations thereof.

20. The method of claim 16, wherein said needle-less injector contains a lyophilized product to mix with said injectate.
